SEQUENCE LISTING (39 sequences)

SEQ ID NO: 1 - protein, 1417 aa, Unknown
  Cas9 protein
MDYKDDDDKDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 2 - DNA, 82 bp, Artificial Sequence
  synthetic WT guide RNA sequence
gttttagagctagaaatagcaagttaaaataaggctagtccgttatcaacttgaaaaagtggcaccgagtcggtgctttttt

SEQ ID NO: 3 - DNA, 25100 bp, Artificial Sequence
  synthetic GST-TAL-FokI-linker-FokI
gcttaagcggtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggccctagagatcacgagactagcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcatcgaattcctgcagcccggtgcatgactaagctagtaccggttaggatgcatgctagctcagttagcctcccccatctctcgacgcggccgctttacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggtggctcgagcggaggctggatcggtcccggtgtcttctatggaggtcaaaacagcgtggatggcgtctccaggcgatctgacggttcactaaacgagctctgcttatataggcctcccaccgtacacgcctaccctcgagaagcttgatatcactagagctctagtgtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacagtgagctagcgctaccggtcgccacccctaggatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccggcggtagtttaaacatggcttcctcccctccaaagaaaaagagaaaggttagttggaaggacgcaagtggttggtctagagtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcctctagatcccagcctgcaggttcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtgagacggaaatttaataacggcgagataaactttggcgcgcctggcggaggtggaagtgcaggtgctggatccggtagtggctcaggtggtggtggcggttcagctggcgctggaagtggttcaggtagtggaggaggaggcggctctgcaggagcaggctctggctccggatctggaggaggtggcggaagcgctggtgcaggctccggaagcggaagtggagcgatcgcttcccagctagtgaaatctgaattggaagagaagaaatctgaacttagacataaattgaaatatgtgccacatgaatatattgaattgattgaaatcgcaagaaattcaactcaggatagaatccttgaaatgaaggtgatggagttctttatgaaggtttatggttatcgtggtaaacatttgggtggatcaaggaaaccagacggagcaatttatactgtcggatctcctattgattacggtgtgatcgttgatactaaggcatattcaggaggttataatcttccaattggtcaagcagatgaaatgcaaagatatgtcgaagagaatcaaacaagaaacaagcatatcaaccctaatgaatggtggaaagtctatccatcttcagtaacagaatttaagttcttgtttgtgagtggtcatttcaaaggaaactacaaagctcagcttacaagattgaatcatatcactaattgtaatggagctgttcttagtgtagaagagcttttgattggtggagaaatgattaaagctggtacattgacacttgaggaagtgagaaggaaatttaataacggtgagataaacttttagttaattaagaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcttaagcggtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggccctagagatcacgagactagcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcatcgaattcctgcagcccggtgcatgactaagctagtaccggttaggatgcatgctagctcagttagcctcccccatctctcgacgcggccgctttacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggtggctcgagcggaggctggatcggtcccggtgtcttctatggaggtcaaaacagcgtggatggcgtctccaggcgatctgacggttcactaaacgagctctgcttatataggcctcccaccgtacacgcctaccctcgagaagcttgatatcactagagctctagtgtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacagtgagctagcgctaccggtcgccacccctaggatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccggcggtagtttaaacatggcttcctcccctccaaagaaaaagagaaaggttagttggaaggacgcaagtggttggtctagagtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcctctagatcccagcctgcaggttcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtgagacggaaatttaataacggcgagataaactttggcgcgcctggcggaggtggaagtgcaggtgctggatccggtagtggctcaggtggtggtggcggttcagctggcgctggaagtggttcaggtagtggaggaggaggcggctctgcaggagcaggctctggctccggatctggaggaggtggcggaagcgctggtgcaggctccggaagcggaagtggagcgatcgcttcccagctagtgaaatctgaattggaagagaagaaatctgaacttagacataaattgaaatatgtgccacatgaatatattgaattgattgaaatcgcaagaaattcaactcaggatagaatccttgaaatgaaggtgatggagttctttatgaaggtttatggttatcgtggtaaacatttgggtggatcaaggaaaccagacggagcaatttatactgtcggatctcctattgattacggtgtgatcgttgatactaaggcatattcaggaggttataatcttccaattggtcaagcagatgaaatgcaaagatatgtcgaagagaatcaaacaagaaacaagcatatcaaccctaatgaatggtggaaagtctatccatcttcagtaacagaatttaagttcttgtttgtgagtggtcatttcaaaggaaactacaaagctcagcttacaagattgaatcatatcactaattgtaatggagctgttcttagtgtagaagagcttttgattggtggagaaatgattaaagctggtacattgacacttgaggaagtgagaaggaaatttaataacggtgagataaacttttagttaattaagaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 4 - DNA, 306 bp, Artificial Sequence
  synthetic nucleotide linker sequence
cctagggggggagggtccggcggcggttccggcggaggatcgggtggagggtcaggtggaggctcaggcggtggatcaggaggagggagcggtggcgggagcggcggagggtcgggaggaggttcgggcggaggctcgggcggtgggtccggaggtggctcgggaggcggaagcggaggcgggtccggtggcggatcaggcggaggcagcggaggaggatcaggtggcggaagcggaggcggctccggaggaggctccggcggtggaagcggtggaggaagcggcggcggatcgggaggtgggtcg

SEQ ID NO: 5 - protein, 102 aa, Artificial Sequence
  synthetic protein linker sequence
PRGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 6 - DNA, 180 bp, Artificial Sequence
  synthetic linker nucleotide sequence
ggcggaggtggaagtgcaggtgctggatccggtagtggctcaggtggtggtggcggttcagctggcgctggaagtggttcaggtagtggaggaggaggcggctctgcaggagcaggctctggctccggatctggaggaggtggcggaagcgctggtgcaggctccggaagcggaagtgga

SEQ ID NO: 7 - protein, 60 aa, Artificial Sequence
  synthetic linker protein sequence
GGGGSAGAGSGSGSGGGGGSAGAGSGSGSGGGGGSAGAGSGSGSGGGGGSAGAGSGSGSG

SEQ ID NO: 8 - protein, 15 aa, Artificial Sequence
  synthetic linker sequence
  MOD_RES (5)..(15): Xaa may be present or absent; if present, repeats as 5 amino acids at a time with a sequence of Gly Gly Gly Gly Ser
GGGGSXXXXXXXXXX

SEQ ID NO: 9 - protein, 27 aa, Artificial Sequence
  synthetic zinc finger motif
  MOD_RES (1)..(27): Xaa is any amino acid
  MOD_RES (6)..(7): Xaa may be present or absent; if present, both residues are present
  MOD_RES (25)..(26): Xaa may be present or absent
XXCXXXXCXXXXXXXXXXXXHXXXXXH

SEQ ID NO: 10 - RNA, 100 nt, Artificial Sequence
  synthetic Cas9gRNA target sequence
  misc_feature (1)..(20): n is a, c, g or u
  misc_feature (24)..(25): n is u for both ribonucleosides or g for both ribonucleosides
nnnnnnnnnnnnnnnnnnnnguunnagagcuagaaauagcaaguuaammuaaggcuaguccguuaucaacuugaaaaaguggcaccgagucggugcuuuu

SEQ ID NO: 11 - DNA, 12 bp, Artificial Sequence
  synthetic nucleotide sequence
ccataaagtagg

SEQ ID NO: 12 - DNA, 17 bp, Artificial Sequence
  synthetic nucleotide sequence
ccataaaggatagtagg

SEQ ID NO: 13 - DNA, 16 bp, Artificial Sequence
  synthetic nucleotide sequence
ccataaagcgagtagg

SEQ ID NO: 14 - DNA, 18 bp, Artificial Sequence
  synthetic nucleotide sequence
ccataaagaccaagtagg

SEQ ID NO: 15 - DNA, 20 bp, Artificial Sequence
  synthetic nucleotide sequence
ccataaagcccccaagtagg

SEQ ID NO: 16 - DNA, 19 bp, Artificial Sequence
  synthetic nucleotide sequence
ccataaggcttaaagtagg

SEQ ID NO: 17 - DNA, 11 bp, Artificial Sequence
  synthetic recombination sequence
cgtgtcgatcg

SEQ ID NO: 18 - DNA, 11 bp, Artificial Sequence
  synthetic recombination sequence
gcgcgtgcaac

SEQ ID NO: 19 - DNA, 13 bp, Artificial Sequence
  synthetic recombination sequence
cgtgtcgatcggc

SEQ ID NO: 20 - DNA, 13 bp, Artificial Sequence
  synthetic recombination sequence
gcgcctcgacacg

SEQ ID NO: 21 - DNA, 96 bp, Artificial Sequence
  synthetic nucleotide sequence
  misc_feature (85)..(86): N is absent at 85-86; Or N at 85 is C and at 86 is T
caccctaactgtaaagtaattgtgtgttttgagactataagtatccctaggagaaccaccttgttggtagcttctgggcgagttnntacgggttag

SEQ ID NO: 22 - DNA, 100 bp, Artificial Sequence
  synthetic nucleotide sequence
  misc_feature (85)..(90): N is absent at 85-90; Or N at 85 is C, at 86 is T, and is C at each of 87-90
caccctaactgtaaagtaattgtgtgttttgagactataagtatccctaggagaaccaccttgttggtagcttctgggcgagttnnnnnntacgggttag

SEQ ID NO: 23 - DNA, 100 bp, Artificial Sequence
  synthetic nucleotide sequence
  misc_feature (84)..(90): N is absent at 84-90; Or N at 84 is C, at 85 is T, at 86-87 is A and at 88-90 is C
accctaactgtaaagtaattgtgtgttttgagactataagtatccctaggagaaccaccttgttggtagcttctgggcgagttnnnnnnntacgggttag

SEQ ID NO: 24 - DNA, 56 bp, Artificial Sequence
  synthetic nucleotide sequence
agtatccctaggagaaccaccttgttggtagcttctgggcgagtttacgggttaga

SEQ ID NO: 25 - DNA, 100 bp, Artificial Sequence
  synthetic nucleotide sequence with barcode
agtatccctaggagaaccaccttgttggtagcttctgggcgagttgctccctcgtgcgctccacctgttccgacccttccggttgccggtacgggttaga

SEQ ID NO: 26 - DNA, 51 bp, Artificial Sequence
  synthetic nucleotide sequence
acgggttagagctagaaatagcaagttaacctaaggctagtccgttatcaa

SEQ ID NO: 27 - DNA, 100 bp, Artificial Sequence
  synthetic nucleotide sequence with barcode
atccctaggagaaccaccttgttggtagcttctgggcgagttagaagctacgggttagagctagaaatagcaagttaacctaaggctagtccgttatcaa

SEQ ID NO: 28 - DNA, 30 bp, Artificial Sequence
  synthetic nucleotide sequence
ccctggtgaaccgcatcgagctgaagggca

SEQ ID NO: 29 - DNA, 22 bp, Artificial Sequence
  synthetic nucleotide sequence with deletion
ccctggtgaaccgcatcgagca

SEQ ID NO: 30 - DNA, 37 bp, Artificial Sequence
  synthetic nucleotide sequence with barcode
ccctggtgaaccgcatcgagcaggggcccgaagggca

SEQ ID NO: 31 - DNA, 40 bp, Artificial Sequence
  synthetic nucleotide sequence
ttggtagcttctgggcgagtttacgggttagagctagaaa

SEQ ID NO: 32 - DNA, 32 bp, Artificial Sequence
  synthetic nucleotide sequence with deletion
ttggtagcttctgtacgggttagagctagaaa

SEQ ID NO: 33 - DNA, 53 bp, Artificial Sequence
  synthetic nucleotide sequence with barcode
ttggtagcttctgggccctcggcctcgagtttcttacgggttagagctagaaa

SEQ ID NO: 34 - DNA, 13 bp, Artificial Sequence
  synthetic nucleotide sequence of barcode insertions
agaagttaaaagt

SEQ ID NO: 35 - DNA, 13 bp, Artificial Sequence
  synthetic nucleotide sequence of barcode insertions
agaagttagaagc

SEQ ID NO: 36 - DNA, 18 bp, Artificial Sequence
  synthetic nucleotide sequence of barcode insertions
agagctacggcttagagc

SEQ ID NO: 37 - DNA, 24 bp, Artificial Sequence
  synthetic nucleotide sequence of barcode insertions
agagctagaaagacgggttagaaa

SEQ ID NO: 38 - DNA, 11 bp, Artificial Sequence
  synthetic nucleotide sequence of barcode insertions
agagttagaaa

SEQ ID NO: 39 - DNA, 19 bp, Artificial Sequence
  synthetic nucleotide sequence of barcode insertions
gagttaccgtaactctggg

(19) United States
(12) Patent Application Publication
(10) Pub. No.: US 2017/0298450 A1
(43) Pub. Date: Oct. 19, 2017

(54) RECONSTRUCTION OF ANCESTRAL CELLS BY ENZYMATIC RECORDING

(71) Applicant: THE REGENTS OF THE UNIVERSITY OF CALIFORNIA, Oakland, CA (US)

(72) Inventor: Michael T McManus, San Francisco, CA (US)

(73) Assignee: THE REGENTS OF THE UNIVERSITY OF CALIFORNIA, Oakland, CA (US)

(21) Appl. No.: 15/509,823

(22) PCT Filed: Sep. 10, 2015
(86) PCT No.: PCT/US2015/049375

§ 371 Date: Mar. 8, 2017

Related U.S. Application Data
(60) Provisional application No. 62/048,695, filed on Sep. 10, 2014.

(51) Int. Cl. C12Q 1/68; C12N 9/22; C12N 15/90; C12N 15/11; C12N 2310/20

(52) U.S. Cl. CPC C12Q 1/6888; C12Y 301/21004; C12N 15/907; C12N 15/11; C12N 9/22; C12N 2310/20

(57) ABSTRACT

Provided herein are compositions aid methods for barcoding mammalian cells. The compositions and methods provided herein further provide methods for tracing such barcoded cells ex vivo or in vivo during the life time of an organism. In one aspect, a method of forming a barcoded cell is provided. The method includes expressing in a cell a heterologous cleaving protein complex including a sequence-specific DNA-binding domain and a nucleic acid cleaving domain. The sequence-specific DNA-binding domain targets the nucleic acid cleaving domain to a genomic nucleic acid sequence, thereby forming a genomic nucleic acid sequence bound to the heterologous cleaving protein complex.

### CROSS-REFERENCE TO RELATED PATENT APPLICATIONS

The present application claims benefit of priority to U.S. Provisional Patent Application No. 62/048,695, filed Sep. 10, 2014, which is incorporated by referenced for all purposes.

### BACKGROUND OF THE INVENTION

One of the most fascinating aspects of multicellular life is the ability for cells to change their identity. Developmental biologists have spent decades trying to understand this process in plants, fungi, and worms. As early as 1929, Walter Vogt used “vital dyes” to label individual cells in Xenopus frog embryos. The tissue(s) to which the cells contribute would thus be labeled and visible in the adult organism. With this method, Vogt was able to discern migrations of particular cells to their ultimate tissue into which they integrated. The information Vogt gathered from his Xenopus tracing experiments was then used to develop early qualitative fate maps for a 32 cell blastula. In 1983, using microscopy, Sulston and colleagues reconstructed an entire C. elegans fate map, in which the lineage of its invariable 959 somatic cells was visibly charted. This was a tremendous milestone for the developmental biology field and the Nobel Prize was awarded in 2002 for this achievement. Yet worms are transparent, and extending this brute force fate mapping method to most other species is not possible.

In 2007 Jeff Lichtman and Joshua Sanes developed ‘Brainbow’ technology, based on transgenic animals harboring Cre recombinase and a multicolor cassette (FIG. 3). While earlier labeling techniques allowed for the mapping of only a handful of cells, Brainbow allows the generation of transgenic reporter mice where more than 100 differently mapped neurons can be simultaneously and differentially illuminated. However the use of Brainbow in the mouse is hampered by the incredible diversity of neurons of the CNS. The sheer cellular density combined with the presence of long tracts of axons make viewing larger regions of the CNS with high resolution difficult. Although this cutting-edge technology is fantastic for microscopically visualizing subsets of related cells, it comes up short for simultaneously and definitively mapping large populations of cells in complex tissues.

Some of the main limitations of all lineage tracing approaches is that of granularity and depth. Granularity is a major limitation when one considers that cell development does not proceed along a linear path, but instead branches out, splaying to many cell types, DNA barcodes have been used to mark lineages, but don't maintain a granular code between different cell types. For example, marking a single hematopoietic stem cell with a single DNA bar code. Every hematopoietic cell in the entire lineage will contain that very same mark. Such an approach may be useful for comparing the competition for hematopoietic reconstitution but it gives no granularity to the individual cells, much less the major and minor branched lineages. Currently there are no approaches for applying unique marks to individual cells in a way that would trace their individual fates. The methods and compositions provided herein solve this and other problems in the art.

### BRIEF SUMMARY OF THE INVENTION

In one aspect, a method of forming a barcoded cell is provided. The method includes in step (i) expressing in a cell a heterologous cleaving protein complex including a sequence-specific DNA-binding domain and a nucleic acid cleaving domain. The sequence-specific DNA-binding domain targets the nucleic acid cleaving domain to a genomic nucleic acid sequence, thereby forming a genomic nucleic acid sequence bound to the heterologous cleaving protein complex. In step (ii) a double-stranded cleavage site is introduced in the genomic nucleic acid sequence bound to the heterologous cleaving protein complex, thereby forming a double-stranded cleavage site in the genomic nucleic acid sequence. In step (iii) random nucleotides are inserted at the double-stranded cleavage site, thereby forming the barcoded cell.

In another aspect, a recombinant cleaving ribonucleoprotein complex including (i) a sequence-specific DNA-binding RNA molecule and (ii) a nucleic acid cleaving domain is provided, wherein the RNA molecule includes a nucleic acid cleaving domain recognition site.

In another aspect, a method of forming a barcoded cell said method is provided. The method includes in step (i) expressing in a cell a recombinant cleaving ribonucleoprotein complex as provided herein including embodiments thereof. The sequence-specific DNA-binding RNA molecule targets the nucleic acid cleaving domain to a genomic nucleic acid sequence, thereby forming a genomic nucleic acid sequence bound to the recombinant cleaving ribonucleoprotein complex. In step (ii) a double-stranded cleavage site is introduced in the genomic nucleic acid sequence bound to the recombinant cleaving ribonucleoprotein complex, thereby forming a double-stranded cleavage site in the genomic nucleic acid sequence. In step (iii) the recombinant DNA editing protein is targeted to the double-stranded cleavage site such as the DNA editing protein inserts a barcoded nucleic acid sequence into the double-stranded cleavage site; thereby forming the barcoded cell.

In another aspect, a recombinant DNA editing protein is provided. The recombinant DNA editing protein includes (i) a sequence-specific DNA-binding domain and (iii) terminal deoxynucleotidyl transferase domain.

In another aspect, a recombinant cleaving protein is provided. The recombinant cleaving protein includes (i) a cell cycle regulated domain, (ii) a sequence-specific DNA-binding domain and (iii) a DNA cleaving domain, wherein the cell cycle regulated domain is operably linked to one end of the sequence-specific DNA-binding domain and the DNA cleaving domain is linked to the other end of the sequence-specific DNA-binding domain.

In another aspect, a recombinant DNA editing protein is provided. The recombinant DNA editing protein includes (i) a cell cycle regulated domain, (ii) a sequence-specific DNA-binding domain and (iii) a terminal deoxynucleotidyl transferase domain, wherein the cell cycle regulated domain is operably linked to one end of the sequence-specific DNA-binding domain and the terminal deoxynucleotidyl transferase domain is linked to the other end of the sequence-specific DNA-binding domain.

In another aspect, a method of forming a barcoded cell is provided. The method includes (i) expressing in a cell a recombinant cleaving protein and a recombinant DNA editing protein in a cell cycle-dependent manner. In step (ii) the recombinant cleaving protein is targeted to a genomic nucleic acid sequence, thereby introducing a double-stranded cleavage site in the genomic nucleic acid sequence. In step (iii) the recombinant DNA editing protein is targeted to the double-stranded cleavage site such as the recombinant DNA editing protein inserts a barcoded nucleic acid sequence into the double-stranded cleavage site; thereby forming the barcoded cell.

In another aspect, a method of forming a barcoded cell is provided. The method includes in step (i) expressing in a cell a recombinant cleaving protein as provided herein including embodiments thereof and a recombinant DNA editing protein as provided herein including embodiments thereof in a cell cycle-dependent manner. In step (ii) the recombinant cleaving protein is targeted to a genomic nucleic acid sequence, thereby introducing a double-stranded cleavage site in the genomic nucleic acid sequence. In step (iii) the recombinant DNA editing protein is targeted to the double-stranded cleavage site such as the recombinant DNA editing protein inserts a barcoded nucleic acid sequence into the double-stranded cleavage site; thereby forming the barcoded cell.

### BRIEF DESCRIPTION OF THE DRAWINGS

FIG. 1. The Cas9 gRNA complex. This image depicts the Cas9: gRNA complex targeting a stretch of DNA. Pairing of 5′-gRNA sequence with cognate DNA (green) triggers Cas9 to induce double-stranded cleavage of the DNA. Cleavage occurs proximal to the PAM motif, in this case NGG (orange). Converting the gRNA stem base to two G:C pairs should result in a self-targeting gRNA which (if active) will destroy itself. Normally this is an unwanted activity, but it will allow Applicants to identify the active gRNAs by deep sequencing the gRNA sequence.

FIG. 2. Barcoding Schematics. A, Two plasmids were designed with the aim to introduce barcodes into cells. The first vector (left hand vector) contains puromycin, mcherry and Cas9 separated by T2A elements. The second vector (right hand vector) contains a self-editing guide RNA driven by a U6 vector, and a separate promoter driving hygromycin T2A CD4 cassette. Cells expressing both plasmids will result in a charged Cas9 guide RNA complex. Pairing of the 5′-gRNA sequence with cognate DNA (green) triggers Cas9 to introduce a double stranded break 3 nucleotides upstream of the PAM sequence in orange (NGG). The schematic displays the new PAM motif introduced into the guide RNA, which will be cut by Cas9 and barcodes will be introduced at this site.

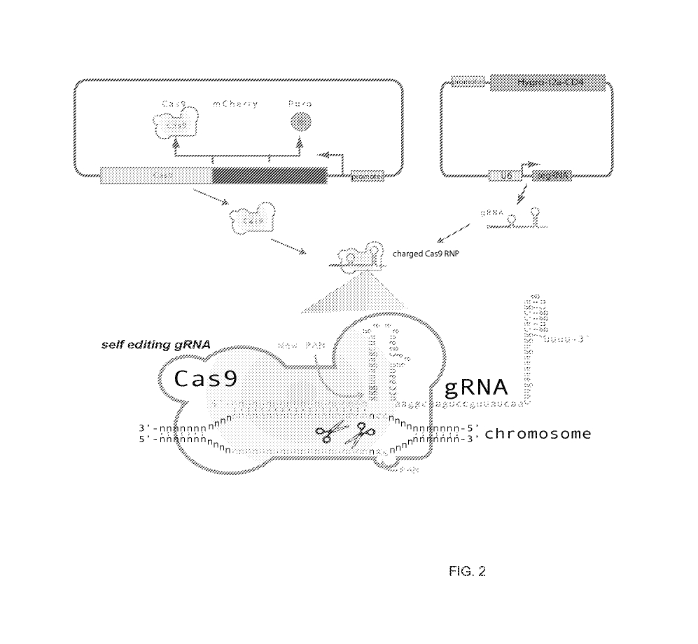

FIG. 3. (A) Brainbow-mouse. Different colors are generated upon random recombination of three spectrally distinct fluorescent proteins. Images show combinatorial expression in the brain (Livet et al., 2007). (B) Confetti-Mouse. A Brainbow construct modified such that Cre deletion removes a stop cassette, resulting in four possible recombination outcomes (image shows small intestine; Snippert et al., 2010b). Although fluorescent is the primary readout, the random recombination provides a short theoretical barcode. (C) illustration that depicts how mixing fluorescent markers may result in a limited number of microscopically discernible cells.

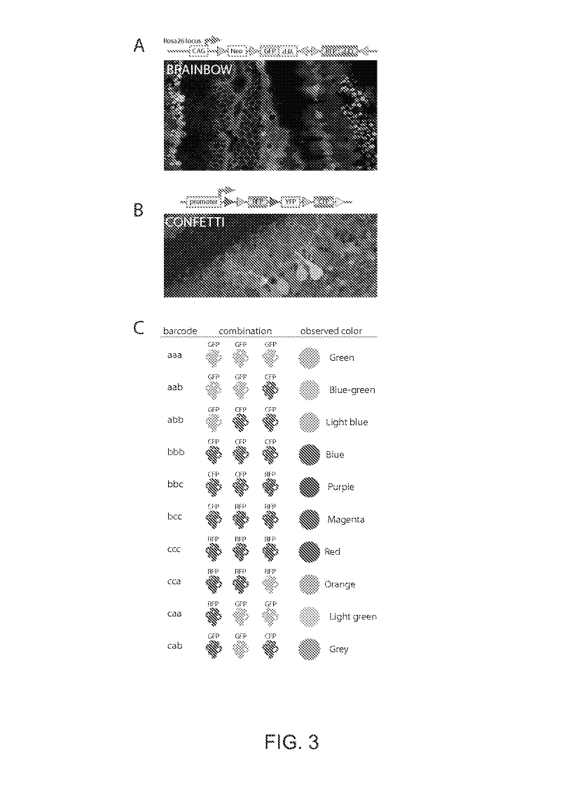

FIG. 4. The tRACER concept. This overview schematic is described in the text. Note that the DNA binding domains of the TALEN:TYPER pair may be immediately side-by-side (proximal) or overlapping (competitive) as shown here. Also, the growing barcode extends away from the TALEN: TYPER pair. The cartoon displays barcode 3mer barcodes, but Applicants will optimize for longer 10-20mer barcodes.

FIG. 5. Single-chain FokI can efficiently cleave DNA. (left) Schematic representation of AZP-scFokI. (right) in vitro activity of a AZP-scFokI variant containing a flexible (GGGGS)¹² linker; lane 1: ctrl DNA substrate, lane 2: incubation with AZP-scFokI. Site-specific cleavage by AZP-scFokI produces 0.9- and 2-kbp DNA fragments (indicated as P1 and P2, respectively). S: a plasmid substrate. FIG. adapted after Mino et al³.

FIG. 6. Modified TALEN and TYPER enzymes. This figure depicts schematics for some of the constructs Applicants have created and are now testing. CC, cell cycle peptide; TAL, TAL effector DNA binding domain; arm, extension peptide; RE, restriction enzyme; SCL, single-chain linker; TdT, terminal deoxynucleotidyl transferase.

FIG. 7. Examples of TdT activity in cultured cells. These preliminary data are derived from transient transfection of cells with a Cas9 targeting nuclease—without (control, ctrl) and with a wild-type TdT cDNA vector (TdT). Image shows a PCR product smear that appears only in TdT transfected cells. The PCR products were cloned, and sequenced (alignment, see right). Green nucleotides are non-templated additions. The control reactions have deletions but no additions.

FIG. 8. Characterization of a Fluorescent Indicator for Cell-Cycle Progression (A) A fluorescent probe that labels individual G₁ phase nuclei in red and S/G₂/M phase nuclei green. (F) Typical fluorescence images of HeLa cells expressing mKO2-hCdt1 (30/120) and mAG-hGem (1/110) and immunofluorescence for incorporated BrdU at G₁, G₁/S, S, G₂, and M phases. The scale bar represents 10 μm. Figure and legend adapted from Miyawaki et al¹.

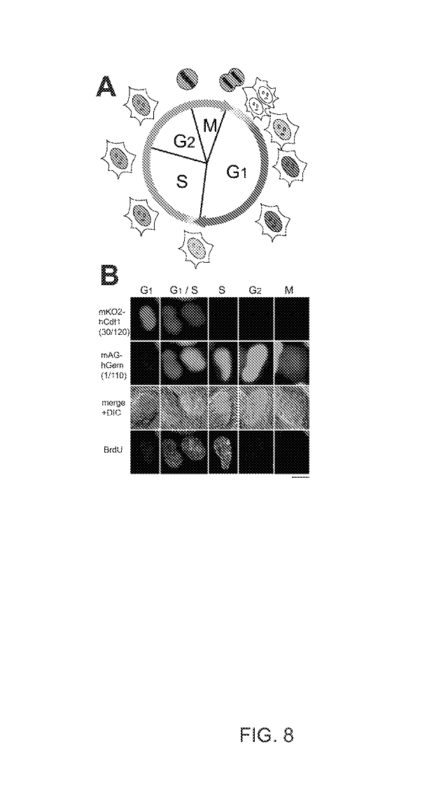

FIG. 9. The tRACER concept is based on naturally occurring phenomenon. VDJ recombination (left) and RNA editing (right) both use cascades of cleavage, terminal transferase activities, and ligation.

FIG. 10. tRACER path. This grossly simplified tracing of the lineage path of a single cell depicts nascent barcodes across the initial eight generations

FIG. 11. New technologies offer tRACER a chance to profile specific cell types in biological settings. LEFT: In situ deep sequencing. Image adapted from Ke et al². RIGHT: Merged brightfield and fluorescence image of microfluidic “cell drops”, showing successful detection of PTPRC via TaqMan probe (red) detection of Raji (green), but not PC3 cells (blue). These are cutting-edge methods that will be married to tRACER, providing spatial resolution and cell-identity to complex phylogenetic mapping experiments

FIG. 12: Schematic representation of embodiments of recombinant DNA editing proteins. Outlined are all constructs that will be generated including combinations of DNA editing enzymes coupled to fluorescent markers, DNA polymerases and ligases.

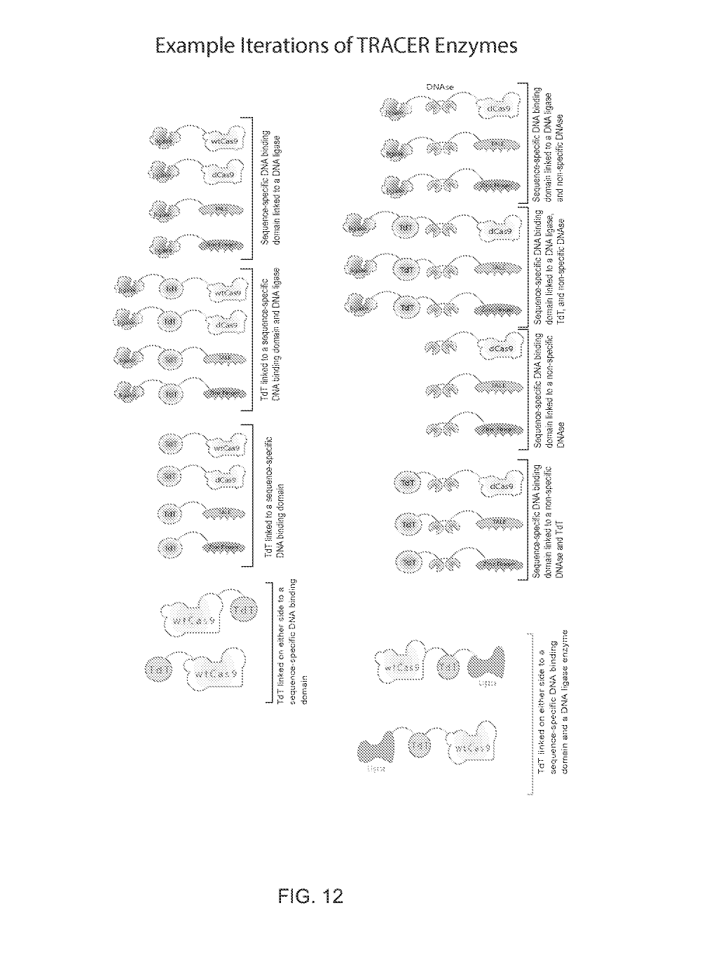

FIG. 13: Schematic representation of a method of forming a barcoded cell.

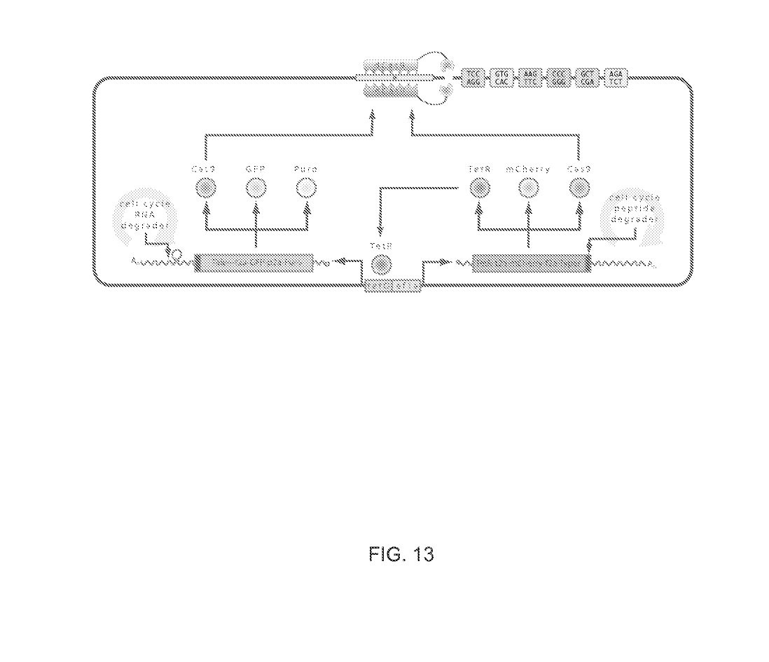

FIG. 14: Evidence of Barcoding in vitro. A, HEK 293 cells were stably transduced with lentiviral construct expressing the self-editing guide RNA. Cells were selected for it week with hygromycin (100 g/ml). Cells were transduced with a lentiviral construct expressing TNT and selected with Zeomycin for 1 week (100 g/ml). Finally cells were transduced with a lentiviral construct expressing Cas9 followed by selection for 1 week with blasticidin (10 g/ml), B, Following 2 weeks of blasticidin selection of the HEK293/Cas9/self-editing guide/TdT cells genomic DNA was extracted and PCR was carried out to amplify the region of interest (left panel). The 250 bp band was gel extracted and TOPO cloned. Colonies were sequenced and barcodes were identified (right panel).

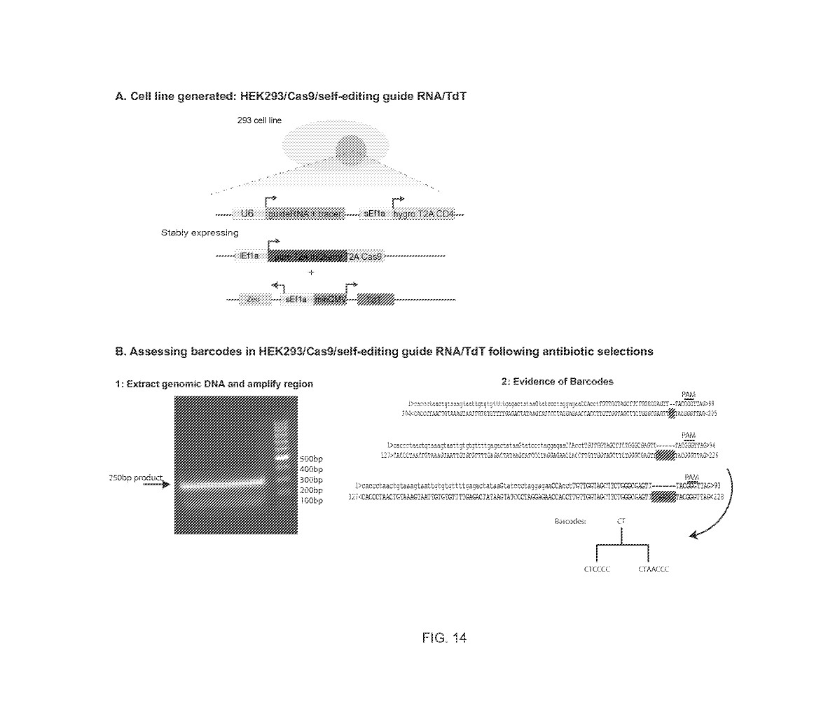

FIG. 15: Evidence of Barcoding in vitro. A, FMK 293 cells were stably transduced with lentiviral construct expressing the self-editing guide RNA. Cells were selected for 1 week with hygromycin (100 g/ml). Cells were transiently transfected with a construct expressing Cas9 fused to GET and linked with TdT. B, 9 days following transfection, HEK293/self-editing guide cells were sorted upon level of gfp expression. Genomic DNA was extracted from gfp positive cells and PCR was carried out, to amplify the region of interest (left panel). The 250 bp band was gel extracted and TOPO cloned. Colonies were sequenced and barcodes were identified (right panel).

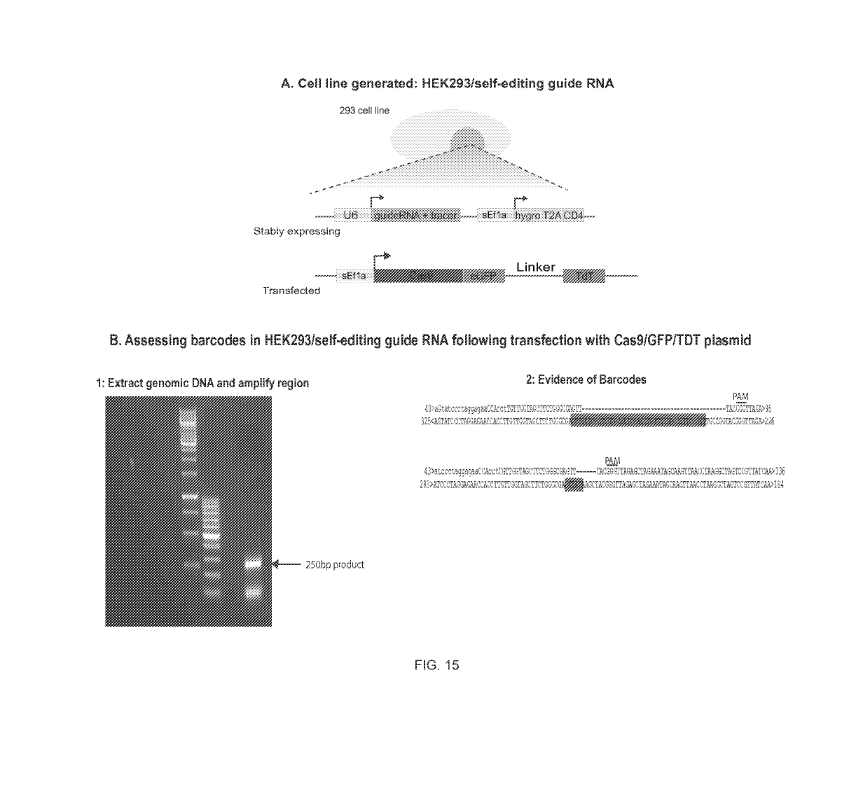

FIG. 16A displays dsDNA break at a conventional DNA locus. FIG. 16B displays a self-editing gRNA (segRNA) locus.

FIG. 17 displays exemplary sequencing results of barcode insertions from terminal transferase.

FIG. 18 depicts constructs introduced into 293T cells.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities fir the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Set. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins. Software for performing BLAST analyses is publicly available through the National Center for Biotechnology Information, as known in the art. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score hills off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (F) of 10, and the BLOSUM62 scoring matrix (see Henikoff & Henikoff, Proc. Natl. Acad. Sci. USA 89:10915 (1989)) alignments (B) of 50, expectation (F) of 10, M=5, N=−4, and a comparison of both strands.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The following eight groups each contain amino acids that are conservative substitutions tier one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (Methionine (M), Valine (V); 6) Phenylalanine (F), Tyrosine (Y), Tryptophan (W); 7) Serine (5), Threonine (T); and 8) Cysteine (C), Methionine (M) (see, e.g., Creighton, Proteins (1984)).

The “active-site” of a protein or polypeptide refers to a protein domain that is structurally, functionally, or both structurally and functionally, active. For example, the active-site of a protein can be a site that catalyzes an enzymatic reaction, i.e., a catalytically active site. An enzyme refers to a domain that includes amino acid residues involved in binding of a substrate for the purpose of facilitating the enzymatic reaction. Optionally, the tem active site refers to a protein domain that binds to another agent, molecule or polypeptide. For example, the active sites of SENP1 include sites on SENP1 that bind to or interact with SUMO. A protein may have one or more active-sites.

Nucleic acid is “operably linked” when it is placed into a functional relationship with another nucleic acid sequence. For example, DNA for a presequence or secretory leader is operably linked to DNA for a polypeptide if it is expressed as a preprotein that participates ire the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

“Heterologous”, when used with reference to portions of a protein, indicates that the protein comprises two or more domains that are not found in the same relationship (e.g., do not occur in the same polypeptide) to each other in nature. Such a protein, e.g., a fusion protein, contains two or more domains from unrelated proteins arranged to make a new functional protein. Similarly, when used in the context of two substances (e.g., nucleic acids, cells, proteins), the two substances are not found in the same relationship to each other in nature. As an example, a “cell expressing a heterologous protein” refers to a cell that expresses a protein that does not naturally occur in the cell.

“Domain” refers to a unit of a protein or protein complex, comprising a polypeptide subsequence, a complete polypeptide sequence, or a plurality of polypeptide sequences where that unit has a defined function.

For specific proteins described herein (e.g., Cas 9, FokI, MmeI), the named protein includes any of the protein's naturally occurring forms, or variants that maintain the protein transcription factor activity (e.g., within at least 50%, 80%, 90%, 95%, 96%, 97%, 98%, 99% or 100% activity compared to the native protein). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment thereof.

The term “Cas 9” as provided herein includes any of the CRISPR associated protein 9 protein naturally occurring forms, homologs or variants that maintain the RNA-guided DNA nuclease activity (e.g., within at least 50%, 80%, 90%, 95%, 96%, 97%, 98%, 99% or 100% activity compared to the native protein). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the Cas 9 protein is the protein as identified by the NCBI sequence reference: GI:672234581. In embodiments, the Cas 9 protein is the protein as identified by the NCBI sequence reference KJ796484 (GI:672234581) or functional fragment thereof. In embodiments, the Cas 9 protein includes the sequence identified by the NCBI sequence referencer GI:669193786. In embodiments, the Cas 9 protein has the sequence of SEQ ID NO:1. In embodiments, the Cas-9 protein is encoded by a nucleic acid sequence corresponding to Gene ID KJ796484 (GI:672234581).

The Zinc finger motif will include Cys2His2 motif (X2-C-X2,4-C-X12-H-X3,4,5-H, where X is any amino acid).

### DETAILED DESCRIPTION OF THE INVENTION

Provided herein are compositions and methods for barcoding mammalian cells. The compositions and methods provided herein further provide means for tracing such barcoded cells in vivo during the life time of an organism. For example, in the methods provided a fusion protein including a sequence-specific DNA-binding domain (e.g., a guide RNA or a TAL effector DNA binding domain) and a nucleic acid cleaving domain (e.g., a restriction enzyme) is targeted to a site in the cellular genome to insert a cleavage site in the genome. A DNA editing protein may then be targeted to said cleavage site to insert random nucleotides (barcode) at the site. The DNA editing enzyme could be endogenous or heterologous. When progeny cells are formed, the process of cleavage and random nucleotide insertion is repeated due to the constitutive or cell cycle-specific expression of the sequence-specific DNA-binding domain and nucleic acid cleaving domain. Every time a progeny cell is formed, additional random nucleotides are inserted at the original cleavage site thereby adding new nucleotides to the existing barcode. The newly formed barcode is longer than the original maternal barcode and is specific for each progeny cell. Since the barcode includes the nucleotides of the maternal barcode it can be used to trace back the maternal source of an individual cell thereby characterizing its ancestral lineage.

A. Cleaving Protein Complex

The cleaving protein complex provided herein is a heterologous protein complex including a sequence-specific DNA-binding domain and a nucleic acid cleaving domain. The cleaving protein complex may be a fusion protein where the sequence-specific DNA-binding domain and the nucleic acid cleaving domain are directly joined at their amino- or carboxy-terminus via a peptide bond. Alternatively, an amino acid linker sequence may be employed to separate the sequence-specific DNA-binding domain and nucleic acid cleaving domain polypeptide components by a distance sufficient to ensure that each polypeptide folds into its secondary and tertiary structures. Such an amino acid linker sequence is incorporated into the fusion protein using standard techniques well known in the art. Suitable peptide linker sequences may be chosen based on the following factors: (1) their ability to adopt a flexible extended confirmation; (2) their inability to adopt a secondary structure that could interact with the first and second polypeptides; and (3) the lack of hydrophobic or charged residues that might react with the first and second polypeptides. Typical peptide linker sequences contain Gly, Ser, Val and Thr residues. Other near neutral amino acids, such as Ala can also be used in the linker sequence. Amino acid sequences which may be usefully employed as linkers include those disclosed in Maratea et al. (1985) Gene 40:39-46; Murphy et al. (1986) Proc. Natl. Acad. Sci. USA 83:8258-8262; U.S. Pat. Nos. 4,935,233 and 4,751,180, each of which is hereby incorporated by reference in its entirety for all purposes and in particular for all teachings related to linkers. The linker sequence may generally be from 1 to about 50 amino acids in length, e.g., 3, 4, 6, or 10 amino acids in length, but can be 100 or 200 amino acids in length. Linker sequences may not be required when the first and second polypeptides have non-essential N-terminal amino acid regions that can be used to separate the functional domains and prevent steric interference. In some embodiments, linker sequences of use in the present invention comprise an amino acid sequence according to (GGGGs)ₙ. In embodiments, linker sequences of use in the present invention include a protein encoded by the nucleotide sequence of SEQ ID NO:4. In embodiments, linker sequences of use in the present invention include a protein having the sequence of SEQ ID NO:5.

Other chemical linkers include carbohydrate linkers, lipid linkers, fatty acid linkers, polyether linkers, e.g., PEG, etc. For example, poly(ethylene glycol) linkers are available from Shearwater Polymers, Inc. Huntsville, Ala. These linkers optionally have amide linkages, sulfhydryl linkages, or heterobifunctional linkages.

Other methods of joining two heterologous domains include ionic binding by expressing negative and positive tails and indirect binding through antibodies and streptavidin-biotin interactions. See, e.g., Bioconjugate. Techniques, Hermanson, Ed., Academic Press (1996).

Nucleic acids encoding the polypeptide fusions can be obtained using routine techniques in the field of recombinant genetics. Basic texts disclosing the general methods of use in this invention include Sambrook and Russell, Molecular Cloning, A Laboratory Manual (3rd ed. 2001); Krigler, Gene Transfer and Expression: A Laboratory Manual (1990); and Current Protocols in Molecular Biology (Ausubel et al., eds., 1994-1999). Such nucleic acids may also be obtained through in vitro amplification methods such as those described herein and in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al, (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117, each of which is incorporated by reference in its entirety for all purposes and in particular for all teachings related to amplification methods.

Alternatively, the sequence-specific DNA-binding domain and the nucleic acid cleaving domain are expressed as individual proteins encoded by separate nucleic acids and the cleaving protein complex is formed through protein interaction.

The term “nucleic acid cleaving domain” as provided herein refers to a restriction enzyme or nuclease or functional fragment thereof. The terms “restriction enzyme” or “nuclease” have the same ordinary meaning in the art and can be used interchangeably throughout. A nuclease is an enzyme capable of cleaving the phosphodiester bonds between the nucleotide subunits of nucleic acids. Nucleases are usually further divided into endonucleases and exonucleases, although some of the enzymes may fall in both categories. Non-limiting examples of nucleases are deoxyribonuclease and ribonuclease. In embodiments, the nucleic acid cleaving domain includes or is a Cas 9 domain or functional portion thereof. In embodiments, the nucleic acid cleaving domain includes or is a restriction enzyme (e.g., MmeI, FokI) or functional portion thereof. Where the nucleic acid cleaving domain includes a restriction enzyme, the nucleic acid cleaving domain may be a restriction enzyme dimer, wherein two restriction enzymes or functional portions thereof are connected through a single-chain linker. In embodiments, the single-chain linker is encoded by a nucleic acid of SEQ ID NO:6. In embodiments, the single-chain linker has the sequence of SEQ ID NO: 7

The sequence-specific DNA-binding domain as provided herein may include a polypeptide or nucleic acid capable of binding a genomic nucleic acid sequence. Where the DNA-binding domain includes or is a nucleic acid, the nucleic acid may be an RNA molecule capable of hybridizing to the genomic nucleic acid sequence. The RNA molecule may be a guide RNA and the genomic nucleic acid sequence may form part of the gene encoding said guide RNA (guide RNA encoding sequence). Therefore, in embodiments, the guide RNA provided herein binds to a part or entirety of its own gene. In embodiments, the guide RNA includes a nucleic acid cleaving domain recognition site. The term “nucleic acid cleaving domain recognition site” refers to a nucleotide sequence, which forms part of the guide RNA and which is recognized by a nucleic acid cleaving domain (e.g., a nuclease). Where the DNA-binding domain includes a polypeptide, the DNA-binding domain may be a TAL (transcription activator-like) effector DNA binding domain or a zinc finger domain.

B. Recombinant DNA Editing Proteins

As described above, the cleaving protein complex as provided herein is targeted to a genomic nucleic acid sequence by sequence-specific DNA binding and inserts a cleavage site at binding site or in close vicinity thereto. Random nucleotides may be subsequently inserted at the cleavage site by further targeting a DNA editing protein to the cleavage site. A DNA editing protein as provided herein is a polypeptide including a terminal deoxynucleotidyl transferase (TdT) activity. A “terminal deoxynucleotidyl transferase” refers to a specialized DNA polymerase, which catalyzes the addition of nucleotides to the 3′ terminus of a DNA molecule. Unlike most DNA polymerases, it does not require a template. The preferred substrate of terminal deoxynucleotidyl transferase is a 3′-overhang, but it can also add nucleotides to blunt or recessed 3′ ends. In embodiments, the terminal deoxynucleotidyl transferase is the protein as identified by the NCBI sequence reference NM_004088.3. In embodiments, the DNA editing protein is an endogenous DNA editing protein. Where the DNA editing protein is an endogenous DNA editing protein, the DNA editing protein is native to, or originates within, a given cell or organism. In embodiments, the DNA editing protein is a recombinant DNA editing protein. The DNA editing protein as provided herein may include a sequence-specific DNA binding domain and a DNA transferase domain. Where the DNA editing protein includes a sequence-specific DNA binding domain and a DNA transferase domain, the DNA editing protein may be a heterologous protein. The DNA transferase domain may include a terminal deoxynucleotidyl transferase or functional fragment thereof. In embodiments, the DNA transferase domain is a terminal deoxynucleotidyl transferase or functional fragment thereof. The sequence-specific DNA binding domain may be as described above, for example an RNA molecule (e.g., a guide RNA), a TAL (transcription activator-like) effector DNA binding domain or a zinc finger domain.

To provide for regulated expression and activity of the protein cleaving complex and the recombinant DNA editing proteins during cell division, they may be operably linked to a cell-cycle regulated domain. A cell cycle regulated domain may be a peptide that is proteolytically cleaved in a cell-cycle dependent manner to ensure the timely accumulation during the appropriate phase of the cell cycle. Alternatively, the cell-cycle regulated domain is a nucleotide sequence which controls the transcription or RNA turnover of the polynucleotide it is operably linked to. Coupling the protein cleaving complex and the recombinant DNA editing proteins provided herein to cell-cycle regulatory elements provides that barcodes will be added in a temporal manner during cell division. In embodiments, the cell-cycle regulatory element is operably linked to the N-terminal end of the sequence-specific DNA binding domain.

C. Fusion Proteins

As described above the sequence-specific DNA binding domain and the nucleic acid cleaving domain forming the cleaving protein complex may be separately expressed or may form part of a fusion protein. Similarly, the sequence-specific DNA binding domain and the DNA transferase domain forming the DNA editing protein may be separately expressed or may form part of a fusion protein. In embodiments, the fusion protein includes a TAL effector DNA binding domain operably linked to a nucleic acid cleaving domain (e.g., two FokI domains separated by a single chain linker). In further embodiments, the N-terminal end of the TAL effector DNA binding domain is operably linked to a cell-cycle regulated domain and the C-terminal end of the TAL effector DNA binding domain is connected through an extension peptide to the nucleic acid cleaving domain.

In embodiments, the fusion protein includes a TAL effector DNA binding domain operably linked to a DNA transferase domain. In further embodiments, the N-terminal end of the TAL effector DNA binding domain is operably linked to a cell-cycle regulated domain and the C-terminal end of the TAL, effector DNA binding domain is connected through an extension peptide to the DNA transferase domain. In embodiments, the fusion protein includes a zinc finger binding domain operably linked to a DNA transferase domain. The fusion protein provided herein may further include a non-specific DNAse domain connecting the DNA binding domain with the DNA transferase domain. In embodiments, the non-specific DNAse domain is a dimer. Alternatively, the cleaving protein complex and the recombinant DNA editing protein may form a fusion protein. Thus, in embodiments, a fusion protein is formed that includes a Cas9 protein and a terminal deoxynucleotidyl transferase, wherein the Cas9 protein is bound to a guide RNA.

D. Methods of Barcoding a Cell

The compositions and methods provided may be used for barcoding mammalian cells. The compositions and methods provided herein further provide means for tracing such barcoded cells in vivo during the life time of an organism or in vitro in a cell (e.g., cell in a cell culture). For example, in the methods provided a fusion protein including a sequence-specific DNA-binding domain (e.g., a guide RNA or a TAL effector DNA binding domain) and a nucleic acid cleaving domain (e.g., a restriction enzyme) is targeted to a site in the cellular genome to insert a cleavage site in the genome. A DNA editing protein may then be targeted to said cleavage site to insert random nucleotides (barcode) at the site. The DNA editing enzyme could be endogenous or heterologous. When progeny cells are formed, the process of cleavage and random nucleotide insertion is repeated due to the constitutive or cell cycle-specific expression of the sequence-specific DNA-binding domain and nucleic acid cleaving domain. Every time a progeny cell is for additional random nucleotides are inserted at the original cleavage site thereby adding new nucleotides to the existing barcode. The newly formed barcode is longer than the original maternal barcode and is specific for each progeny cell. Using sequencing methodologies well known in the art (e.g., deep sequencing) the barcode sequence of each cell can be identified and its maternal origin determined. Further, applying deconvolution methodology well known in the art and referred to herein, the maternal source of an individual cell can be traced back thereby characterizing its ancestral lineage. References disclosing the general methods of deconvolution include Vogt W. et al. Gastrulation und Mesodermbildung bei Urodelen und Anuren. II. Teil. W. Roux Arch Entwicklungsmech Org 120384-706. Keller R E (1986) Developmental Biology; 1929; Sulston J E et al. The embryonic cell lineage of the nematode Caenorhabditis elegans Developmental Biology 1983 November; 100(1):64-119; Livet J et al. Transgenic strategies for combinatorial expression of fluorescent proteins in the nervous system Nature. 2007; Snippert H J et al. Intestinal Crypt Homeostasis Results from Neutral Competition between Symmetrically Dividing Lgr5 Stem Cells Cell: 2010 October; 143(1):134-44; Mino T et al. Efficient double-stranded DNA cleavage by artificial zinc-linger nucleases composed of one zinc-finger protein and a single-chain FokI dimer Journal of Biotechnology 2009 March; 140(3-4):156-61; Sakaue-Sawano A et al. Visualizing Spatiotemporal Dynamics of Multicellular Cell-Cycle Progression Cell 2008 February; 132(3):487-98; Ke R et al. In situ sequencing for RNA analysis in preserved tissue and cells Nature methods 2013 September; 10(9):857-60; Balzer M A et al. Amplification dynamics of human-specific (HS) alu family members Nucleic Acids Res. Oxford University Press; 1991 July 11; 19(13):3619-23; Ohtsuka E et al. An alternative approach to deoxyoligonucleotides as hybridization probes by insertion of deoxyinosine at ambiguous codon positions Journal of Biological Chemistry American Society for Biochemistry and Molecular Biology; 1985 March 10; 260(5):2605-8; Rossolini G M et al. Use of deoxyinosine-containing primers vs degenerate primers or polymerase chain reaction based on ambiguous sequence information Molecular and Cellular Probes 1994 April; 8(2):91-8; Maratea D et al. Deletion and fusion analysis of the phage φX174 lysis gene E. Gene 1985 January; 40(1):39-46; Murphy J R et al. Genetic construction, expression, and melanoma-selective cytotoxicity of a diphtheria toxin-related alpha-melanocyte-stimulating hormone fusion protein Proc Natl Acad Sci. USA National Acad Sciences; 1986 November; 83(21):8258-62; Kwoh D Y et al. Transcription-based amplification system and detection of amplified human immunodeficiency virus type 1 with a bead-based sandwich hybridization format Proc Natl Acad Sci USA. National Acad Sciences; 1989 February; 86(4):1173-7; Guatelli J C et al. Isothermal, in vitro amplification of nucleic acids by a multienzyme reaction modeled after retroviral replication Proc Natl Acad Sci USA. National Acad Sciences; 1990 March; 87(5):1874-8; Lomeli H et al. Quantitative assays based on the use of replicatable hybridization probes Clinical Chemistry. American Association for Clinical Chemistry; 1989 September; 35(9):1826-31; Landegren U et al. A ligase-mediated gene detection technique Science. American Association for the Advancement of Science; 1988 August 26; 241(4869):1077-80; Wu D Y et al. The ligation amplification reaction (LAR)—Amplification of specific DNA sequences using sequential rounds of template-dependent ligation. Genomics 1989 May; 4(4):560-9; Barringer K J et al. Blunt-end and single-strand ligations by Escherichia coli ligase: influence on an in vitro amplification scheme Gene. 1990 April; 89(1):117-22; Jimënez J I et al. Comprehensive experimental fitness landscape and evolutionary network for small RNA Proc Natl Acad Sci USA National Acad Sciences; 2013 September 10; 110(37):14984-9; Schloss P D et al. Introducing mothur: open-source, platform-independent, community-supported software for describing and comparing microbial communities Appl Environ Microbiol. American Society for Microbiology; 2009 December; 75(23):7537-41; Li W et al. Cd-hit: a fast program for clustering and comparing large sets of protein or nucleotide sequences Bioinformatics 2006; each of which is incorporated by reference in its entirety for all purposes and in particular for all teachings related to amplification methods.

The methods of barcoding a cell provided herein including embodiments thereof may further include a step of ligating the ends of the double-stranded cleavage site. The ligation enzymes used for this ligation step may be endogenous DNA ligation enzymes (e.g., a ligase that naturally occurs in the cell being barcoded). In embodiments, the ligation enzyme is a heterologous DNA ligation complex. A heterologous DNA ligation complex as provided herein includes a sequence-specific DNA-binding domain and a nucleic acid ligation domain. In further embodiments, the heterologous DNA ligation complex includes a DNA editing domain. A DNA editing domain as provided herein includes a protein having terminal deoxynucleotidyl transferase (TdT) activity. Thus, in embodiments, the method further includes after step (iii) of inserting random nucleotides a step (iii.i) of ligating the ends of the double-stranded cleavage site. In embodiments, the ligating is achieved by contacting the double-stranded cleavage site with an endogenous DNA ligase. In embodiments, the ligating is achieved by contacting the double-stranded cleavage site with a heterologous DNA ligation complex. In embodiments, the heterologous DNA ligation complex includes a sequence-specific DNA-binding domain and a nucleic acid ligation domain.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

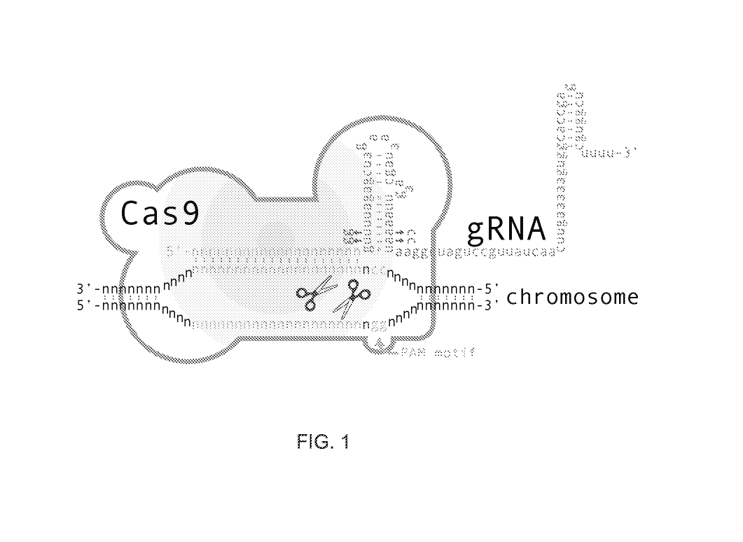

Cas9-based systems potentially represent a significant advance. The prokaryotic CRISPR adaptive immune system has led to the development of custom nucleases whose sequence specificity can be programmed by small RNAs. CRISPR loci are composed of an array of repeats, each separated by ‘spacer’ sequences that match the genomes of bacteriophages and other mobile genetic elements. This array is transcribed as a long precursor and processed within the repeat sequences to generate small crisper RNA (crRNA) that specifies the target dsDNA to be cleaved. An essential feature is the protospacer-adjacent motif (PAM) that is required for efficient target cleavage (FIG. 1). Cas9 is a double-stranded dsDNA endonuclease that uses the crRNA as a guide to specify the cleavage site. To change the target, one only needs to alter the small guiding RNA sequence, a key advantage over TALENs, ZENs, and Megs. For this reason, Applicants' main approach is to develop the Cas9 system for efficient high-throughput gene targeting.

A new approach is provided for tracing the evolutionary history of cells at the most possible granular level, the individual cells. Applicants take advantage of new technologies (deep sequencing and TALENs) combining them in a way to create a single cell lineage tracer in which each cell contains a unique barcode. This system is comprised of a synthetic “TYPER” genetic circuit which can be introduced into cells via homologous recombination or more conveniently, via a retrovirus. Once created, Applicants' vision is to introduce the TYPER circuit into fertilized zygotes, were mouse lines will be developed. In essence every cell in a TYPER mouse will contain a unique barcode, and each barcode would contain information on its previous lineage, starting with the fertilized zygote. This technology, the Reconstruction of Ancestral Cells by Enzymatic Recording (tRACER) is accomplished using two custom enzymes that Applicants have built and are currently optimizing for the digital tracing of cell lineages.

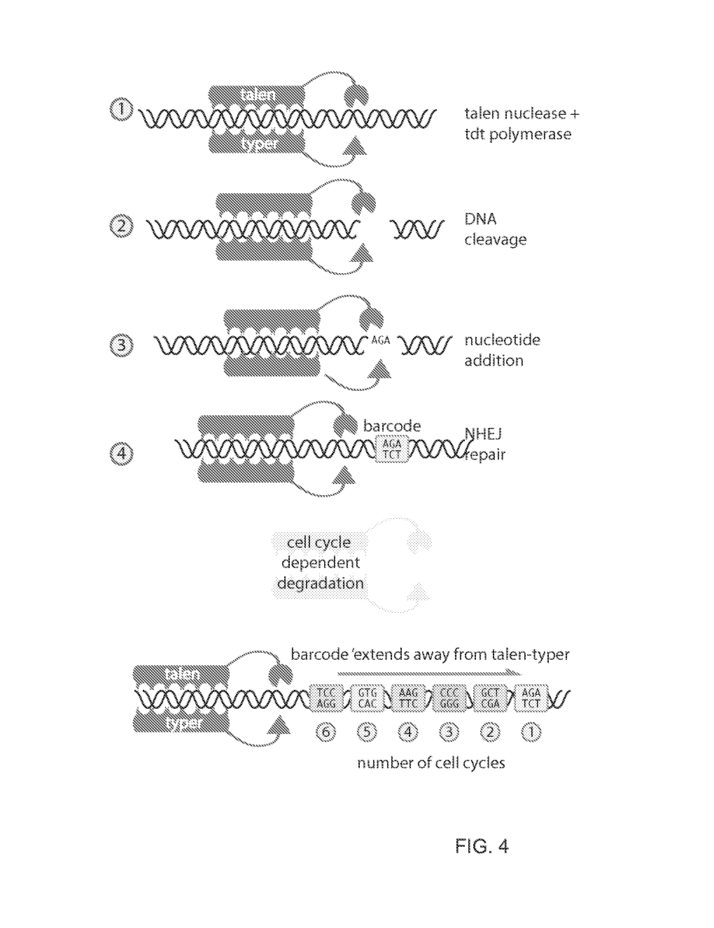

Applicants' first goal is to tangibly realize the concept described in FIG. 4. The foundation of this concept is the development of two distinct enzymes: a modified TALEN and a novel ‘TYPER’. Applicants have recently built these two enzymes and are currently characterizing their activity in vitro and in vivo.

Modified TALENs. Transcription activator-like effector nucleases (TALENs) are essentially artificial restriction enzymes generated by fusing a TAL effector DNA binding domain to a DNA cleavage domain. A simple code between amino acid sequences in the TAL effector DNA binding domain and the DNA recognition site allows for protein engineering applications. This code has been used to design a number of specific DNA binding protein fusions.

TALENs are typically used in pairs, where each TALEN cleaves only a single-strand. In genome engineering applications, TALEN binding sites are designed juxtaposed and proximal, producing double-stranded DNA (dsDNA) cleavage. Notably this offers a higher level of specificity, requiring a collectively longer recognition site. Most importantly, each TALEN is composed of a TAL effector DNA binding domain linked to the FokI restriction enzyme, and the FokI enzyme requires dimerization to produce a dsDNA cleavage.

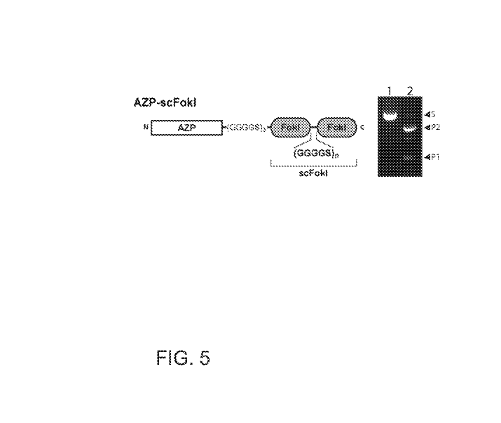

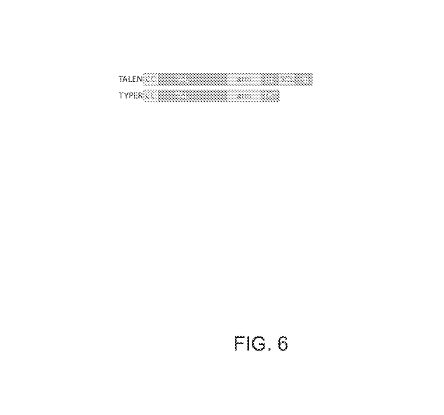

Applicants have recently synthesized novel TALENs designed to cleave both strands. These unique FIG. 5. Single-chain FokI can efficiently cleave DNA. (left) Schematic representation of AZP-scFokI. (right) in vitro activity of a. AZP-scFokI variant containing a flexible (GGGGS) 12 linker; lane 1: ctrl DNA substrate, lane 2: incubation with AZP-scFokI. Site-specific cleavage by AZP-scFokI produces 0.9- and 2-kbp DNA fragments (indicated as P1 and P2, respectively). S: a plasmid substrate. adapted after Mino et al. nucleases are composed of the traditional TAL effector DNA binding domain fused to single a nuclease domain that nicks one DNA strand. However, Applicants have engineered the FokI enzyme as a dimer using a flexible single chain linker, allowing it to cleave dsDNA. Synthetic FokI dimers based on zinc finger DNA binding domains (i.e. not TAL effectors) have been created and contain robust activity in vitro (FIG. 5). Applicants have created 1) a. TAL effector fused to a single-chain FokI, and 2) a TAL effector fused to a single-chain MmeI (FIG. 6). The main difference between these TALENs is the overhang that is produced: FokI produces a four nt 5′-overhang and MmeI produces a two nt 3′-overhang. Applicants' goal is to test and optimize several restriction enzymes when coupled to TAL effector DNA binding domains. Only one enzyme will be needed for the tRACER platform. The ideal enzyme will exhibit maximal activity and specificity on its DNA target site, allowing for robust enzymatic machinations with a novel ‘TYPER’ enzyme Applicants describe below.

A novel TYPER enzyme. Applicants have constructed a unique enzyme fusion between a TAL effector DNA binding domain and a terminal deoxynucleotidyl transferase (TdT) (FIG. 6). TdT is a nuclear enzyme responsible for the non-templated addition of nucleotides at gene segment junctions of developing lymphocytes 4. For B cells and T TdT is a key component of their development, participating in somatic recombination of variable gene segments. Regulated rearrangement of lymphocyte receptor gene segments through recombination expands the diversity of antigen-specific receptors. TdT binds to specific DNA sites, adding non-templated A, T, G, and C nucleotides to the 3′-end of the DNA cleavagesite, and is critical value for antigen-specific receptor diversity. The ability of TdT to randomly incorporate nucleotides greatly aids in the generation the ˜1014 different immunoglobulins and ˜1018 unique T cell antigen receptors.

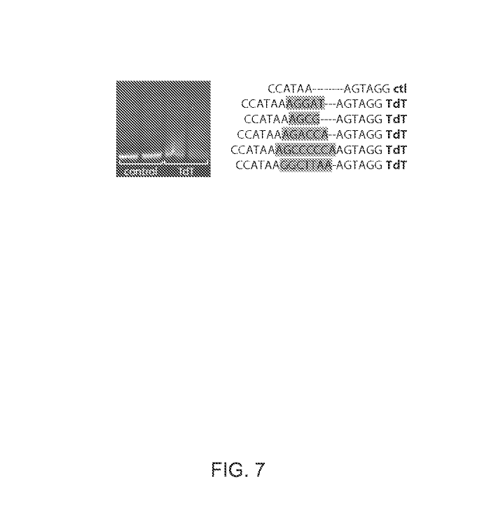

TdT is perhaps the most enigmatic of DNA polymerases, as it bends many of the general rules: not only does it not require a template strand, it does not appear to be processive. Regulated activity at VDJ junctions is limited, typically adding 4-6 nucleotides in a highly regulated process; however, overexpression in non-lymphoid cell lines can yield large insertions (>100 nt) 5, and the recombinant TdT enzyme can robustly add thousands of nucleotides under unregulated conditions. In non-optimized limited cleavage assays Applicants have found that it readily adds up to 4-8 residues to Cas9 induced breakpoints (FIG. 7) and hypothesize it may help ‘lock-in’ Cas9 dsDNA cleavage. Different number of nucleotides may be added when TdT is ‘tethered’ near a DNA 3′-end using a TAL, effector DNA binding domain. Applicants hypothesize that the length of the linker may limit the number of nucleotides added; if so, Applicants will modify the linker domain as needed to change barcode length.

Cell cycle regulation. One aspect of the tRACER system is that it is active during cell division, such that barcodes will be added in a temporal manner. This is not an essential feature of the TRACER technology but may desirably restrict TRACER activity. Cell cycle is a carefully regulated process that ensures DNA replication occurs only once during the cell cycle. In higher eukaryotes such as humans, proteolysis and Geminin (hGem) mediated inhibition of the licensing factor hCdt1 are essential for preventing DNA re-replication. Due to cell cycle-dependent proteolysis, protein levels of hGem and hCdt1 oscillate inversely, with hCdt1 levels being high during G1, while hGem levels are the highest during the S, G2, and M phases. Their regulation is governed by proteolytic rather than transcriptional controls or RNA turnover to ensure the timely accumulation during the appropriate phase. Consistent with this mode of regulation, hGem and hCdt1 peptides can be added onto proteins to regulate their expression in a robust cell-cycle dependent manner. This strategy has been incredibly successful for developing fluorescent markers that definitively illuminate cell cycle progression. To accomplish this Applicants will conjugate hGem peptide sequences onto both the TYPER and TALEN enzymes to pulse-restrict their expression during the cell cycle. If further restriction is needed, Applicants may be able to harness other cell cycle regulatory elements, such as APC^{Cdc20} regulation which is active during M-phase. The general concept is to trigger tRACER TALEN cleavage and TYPER activity only when cell divide. In some embodiments, one can employ cell cycle proteolytic regulation. Optionally, one may also test cycle dependent transcriptional activation/repression or cell RNA turnover. If needed, these regulatory processes might be able to be combined to augment finer restriction of tRACER activity. In some embodiments, an inducible tRACER apparatus could be immensely valuable in pulse-type experiments. This could be made possible by coupling the enzymes to ERT2 or possible placing it in the context of optogenetic regulation.

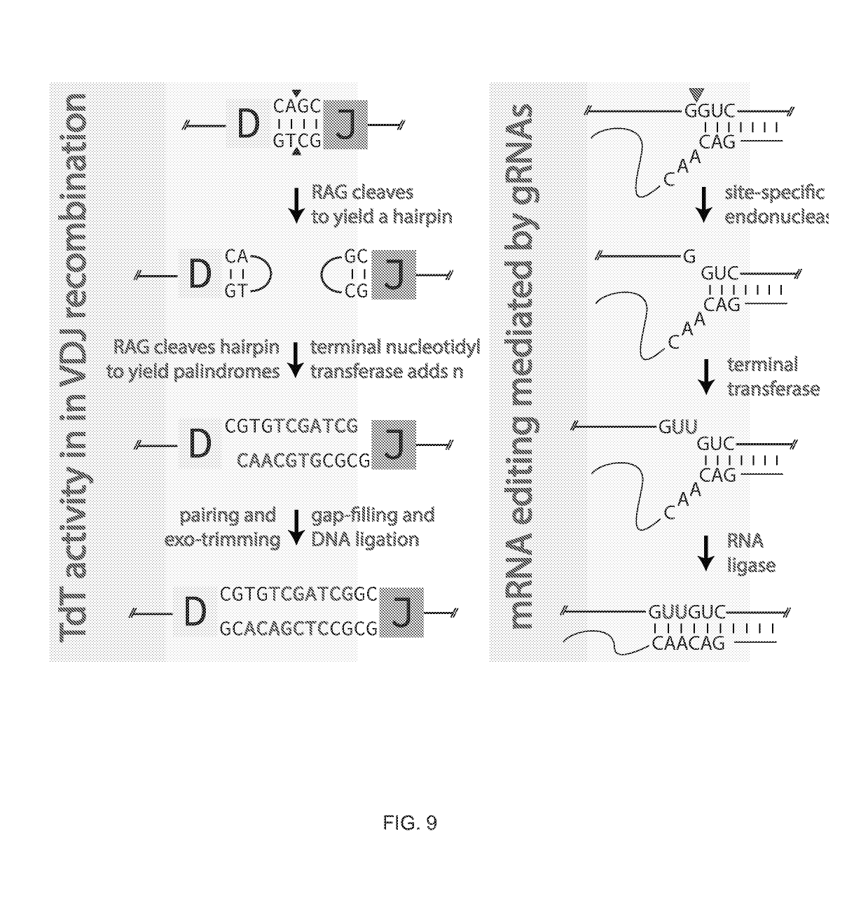

As a general concept, it is worth noting that regulated cycles of nucleic acid cleavage, terminal transferase, and ligation occur in different cell types among different species, including the evolutionarily ancient Trypanosomes (FIG. 9). Another striking example (not depicted here) of regulated retention of DNA ‘barcodes’ at a specific locus is the prokaryotic CRISPR array that provides phage immunity and a long history (many years) of each species subtype.

Bioinformatic considerations. Although Applicants retain flexibility for barcode length, some practical aspects should be considered when optimizing for enzyme activity. A first consideration is that extremely short barcodes may limit the number of cell types that can be analyzed in parallel. However one must consider that if one begins the tRACE with a small number of cells, the second barcode adds to the complexity and allows deconvolution using traditional cladistics analysis (via Bayesian inference of phylogeny). Bayesian inference of phylogeny is based upon the posterior probability distribution of fate map trees, which is the probability of a given phylogenetic tree conditioned on a deep sequencing dataset. Because the posterior probability distribution of trees is impossible to calculate analytically, Markov chain Monte Carlo simulation may be used to approximate the posterior probabilities of trees.

Applicants expect phylogenetic nonconformities and interesting mapping patterns may result from biologic origins, including asymmetric cell division and limited barcoding activity to occur outside of the context of cell division. Similarly Applicants expect nonconformities that result from technical origins such as barcode loss or mutation during the experiment and sample preparation. Notably Applicants do not necessarily need to capture 100% of barcoded cells to reconstruct the cell division tree and assemble testable fate map models. In fact, the resolution depends on the number of cells and the complexity of the trees, a<1% capture rate may be sufficient in many applications, and even less when large numbers of cells are examined.

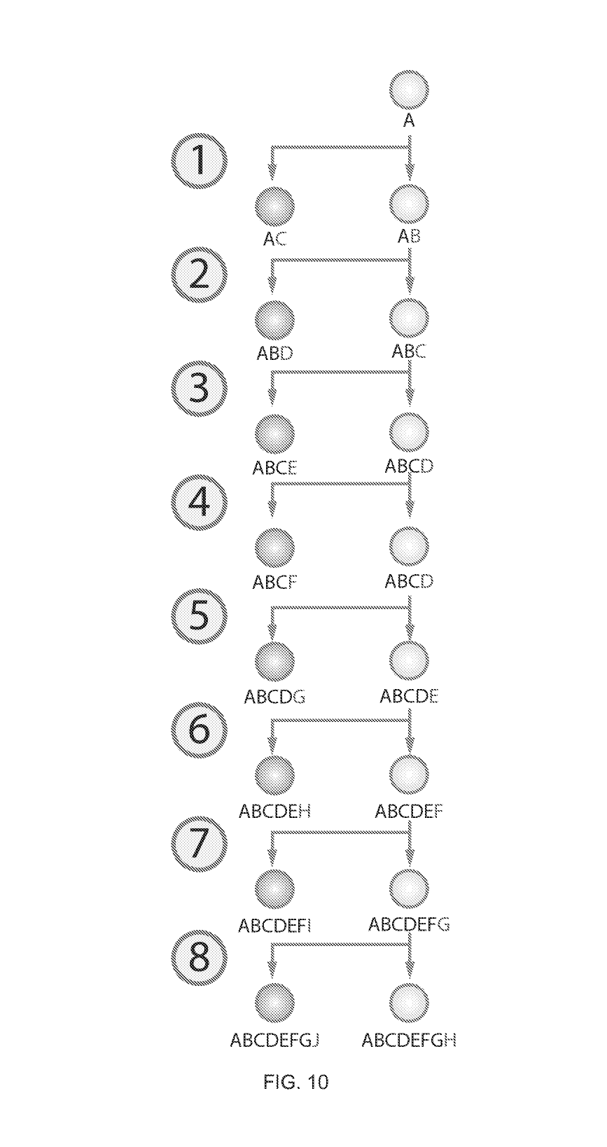

In some embodiments, one can optimize the lengths of the barcodes. While minimal lengths are technically desirable, tone should ensure that the information content is appropriately long enough to uniquely map to a specific cell. In determining the minimal barcode length, a relevant consideration is the number of cells present at the outset of the experiment. Here Applicants would define n as the starting number of unique barcoded cells. Because the barcode history contributes to the growing complexity, in theory a single nucleotide added at each cell doubling would be wholly sufficient, providing you start from a single cell (FIG. 10). However, in practice, limited exonucleolytic trimming during DNA repair would complicate the results. Hence, one goal can be to optimize barcode lengths between 15-20 bp, giving some buffer for potential trimming, and allow one to initiate experiments with extremely large numbers of cells. Limited exonucleolytic trimming of the barcode will simply generate additional uniqueness and should not negatively affect data interpretation.

Statistical considerations. In some embodiments, one can use the Illumina HiSeq 2500, a platform having two general considerations: read length and number of reads. The maximal confidence read length is approximately 200 nt (2×100 bp) hence the combinations of barcodes and their lengths cannot exceed what can be physically read by Illumina sequencing. Depending on barcode length, 200 nt can accommodate 10-50 cell doublings. The Illumina platform has a high output (nearly 3 billion reads per fill run) which is sufficient for focused experiments, but would be no match for the trillions of reads needed to deconvolute an entire mouse, particularly given the need for read redundancy. With these limitations it can be assumed that tRACER could fate map in a single Illumina run approximately at least 10⁷ cells, assuming a 300 fold sequence coverage.

Another consideration is that many parallel internal tRACER ‘biological replicates’ can be obtained in some experimental settings. For example, introducing the construct into mouse ES cells and letting them divide several times in culture will establish ‘pre-barcoded’ cells. Co-injecting 10-12 pre-barcoded tRACER ES cells into a single blastocyst might act as internal replicates, with the potential caveat that some cells may not fully contribute to all lineages. Given the numbers of cells present at gastrulation and shortly thereafter, tRACER is ideal for mapping early and portions of mid-stage mouse embryos.

Tracing space and time. With any DNA modification system, a potential caveat is whether the expression of DNA modifying enzymes would promote tumorigenesis when present in the animal. This has not been observed with TALEN or CRISPR systems but remains a formal possibility. If tumors do appear, their tRACER phylogenetic analysis could prove very interesting in its own right. In fact, the contribution of stem cells to cancer remains a debate. It is unknown whether cancer stem cells are the origin of all malignant cells in the body, and whether they are responsible for the existence of drug-resistant and metastatic cancer cells. tRACER offers a unique opportunity to definitively mark the cell-of-origin for any cancer types.

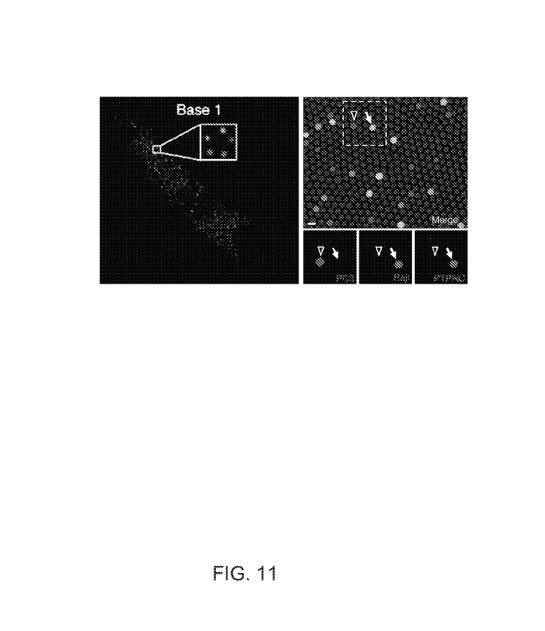

Once tRACER is optimized, Applicants' goal is to integrate spatial and cell-type information. tRACER barcodes do not identify specific cell types but instead generate testable models for uncovering new or pathologically diverged lineages in an ultra high-throughput fashion. However, there are a number of already-developed downstream technologies that allow both spatial and cell-type information will be integrated with tRACER. In some embodiments, one can evaluate whether laser capture of tRACER barcodes from immunohistochemically stained embryonic pancreatic islet cells fate can inform cell origins maps. Such a focused approach will provide both barcode identification and confirmation of specific cell types and their lineages. Second, multiplex FISH will allow probing tissue sections with LNAs against the barcodes. This would allow large numbers of barcodes to be probed simultaneously (using quantum dot or other markers), perhaps in three-dimensional space using whole embryos or whole-mount tissues. Third, an in situ tissue deep sequencing method was recently developed, paving the way for tRACEing hundreds of thousands to millions of immunohistochemically stained cells (FIG. 11, left panel).

Another goal is to integrate tRACER with a novel ultrahighthroughput platform that combines droplet-based microfluidic techniques and PCR to define cell types (FIG. 11, right panel). Applicants' goal is to sort individual cells based on their tRACER barcode and generate RNA-sell libraries. These single-cell RNA-seq libraries can be barcoded and pooled to analyze true single cell gene expression for large numbers of cell types. These systems will give Applicants an unprecedented view of gene expression, digitizing cell identity over developmental space and time.

The adult human body is composed of trillions of cells that all originated from a single fertilized egg cell. In the adult, most tissues are in a state of constant flux, where old cells die and new cells are created from resident populations of stem cells. Disease such as cancer emerges when cells lose their directions, and divide in an uncontrolled manner, losing their identities. Other diseases are hallmarked by a loss of cells, triggered by unwanted self-elimination such as apoptosis or autoimmunity. The fluidity of cell populations initiates from the moment a being is conceived to the being's final breath of life. Multicellular life dances to the music of a highly ordered process, directed by a score that is not well understood.

Cell heterogeneity—inherent differences between individual cells in a given tissue or tumor—is one of the biggest challenges in research today. Current techniques are greatly limited in their ability to mark individual cells while retaining their ancestry. tRACER offers a light year leap. Heterogeneity is a natural consequence of biology, fostering the evolutionary adaptation that hampers cancer treatment.

Using current technologies, it is practically impossible to map the origin of the initial rogue cancer cell that causes a tumor. In essence, using tRACER technology, Applicants will be able to probe the cell of origin of any cancer by deep sequencing the barcodes within a given tumor. Specifically, each cell in that tumor would contain a barcoded digital DNA record of its evolutionary path. Moreover, sequencing barcodes from metastatic cells will trace the cells back to their original tumor and again their wild type healthy cell-of-origin, whether that be a stem cell, a mid-stage progenitor, or a fully differentiated nondividing cell type. Likewise, tracing cell death and amplification in the context of drug treatment may provide information about the evolution of a tumorigenesis during treatment. The origin of cancer heterogeneity has been controversial, with good data to support epigenetic and genetic heterogeneity models. New tools are needed to better understand the origin, development, and evolution of cancers, and the ability to describe tumors at the resolution of single cells could transform one's ability to plot the best treatment options and to anticipate disease outcome.

Currently there are no technologies that can delineate cell ancestries on such a large scale. Applicants' proposed concept takes advantage of the growing power of deep sequencing, as Applicants have the power to sequence billions of reads, potentially tracing hundreds of millions of cells or more. This represents a tremendous step forward from the scale at which fate mapping is currently done (typically qualitatively hundreds of cells).

Derivation and use of a self-editing gRNA for TRACER.

Concept and mechanism of activity. Applicants have developed a novel mechanism for the self-destruction of a gRNA, namely the inclusion of a PAM motif within the context of an actual gRNA (Applicants name self-editing gRNA, or segRNA). Conceptually PAM motifs within the gRNA should be absolutely avoided in natural prokaryotic CRISPR settings as self-destruction would cause loss of CRISPR function and worse, genome instability. However Applicants have found that the tracer portion of the gRNA can be altered to include a PAM motif; Applicants have discovered that the DNA encoding that specific gRNA can be recognized by the gRNA to which it encodes. In this way, the PAM motif causes a self-destruction of the gRNA guiding portion. A precept of the segRNA is that it does not necessarily destroy the upstream promoter that transcribes it, nor the downstream tracer portion of the gRNA that is important for Cas9 binding.

Definition of self-editing. Self-editing occurs when the gRNA has successfully cut its own gene. In the TRACER system, the TdT will add nucleotides to the cut-site, resulting in a change in the DNA guiding portion of the gRNA (depicted in green in FIG. 1). This could be one nucleotide or more that is added, but importantly should have enough added nucleotides to specify the cell lineages within a given experiment.

Promoter and relevance of transcription. In principle the promoter can be poi II or pol III or perhaps pol I. The key element to consider is that the gRNA, once self-edited, will continue to be transcribed, allowing for new gRNAs to be created and destroy the new self-edited gRNA gene. It is in fact an ever-changing process where repeating cycles of self-editing give rise to new gRNA genes which give rise to new gRNA transcripts that self edit.

Length of barcode. Applicants expect that each cycle of self-editing will cause multiple nucleotides being added within a given cell. Applicants are working on regulating the cell-cycle nature of this process, but reason that it does not necessarily need to be cell cycle regulated. The important concept is that the nascent barcodes are unique for a given cell, no matter how or when they are added. Since the barcodes are not ‘forgotten’, new cell divisions give rise to new barcodes which extend the length of the barcode array (FIG. 4).

Applicants' current system allows for the barcode array to be compact, allowing for sequencing of the array by Illumina sequencing, effectively giving billions of reads. Longer reads can be achieved by PacBio technologies.

### Example 2

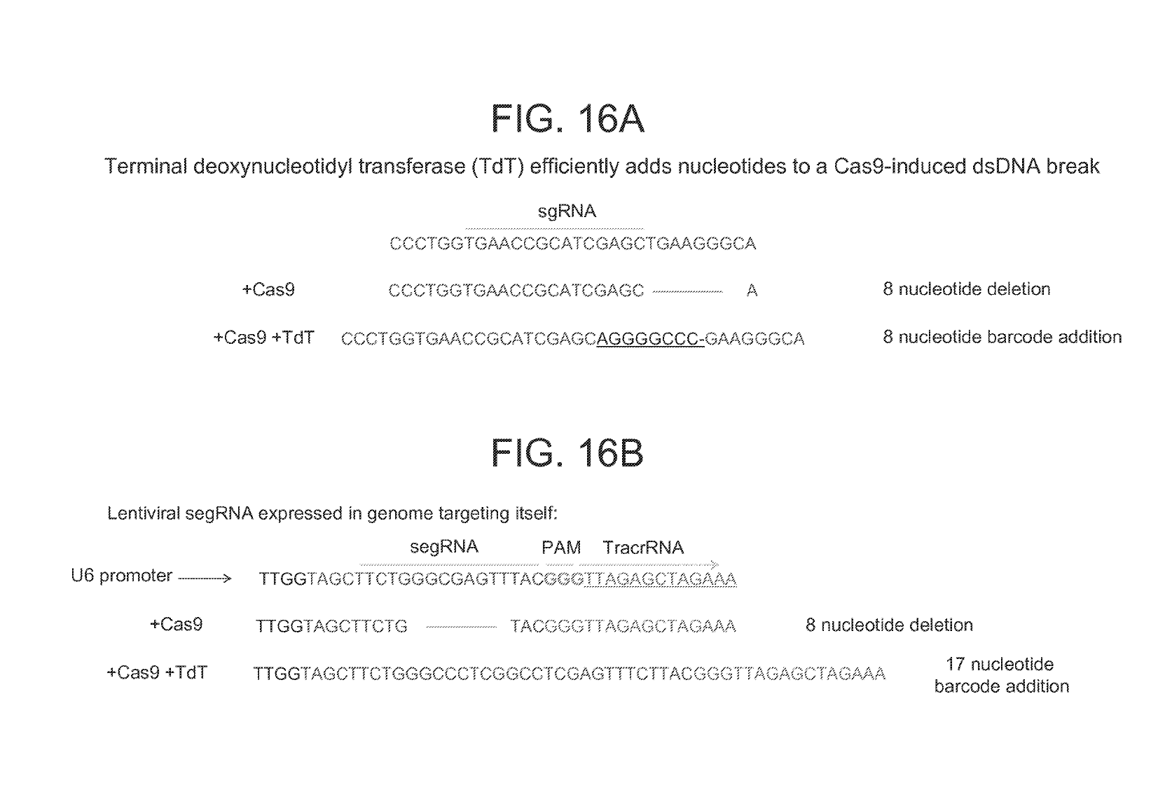

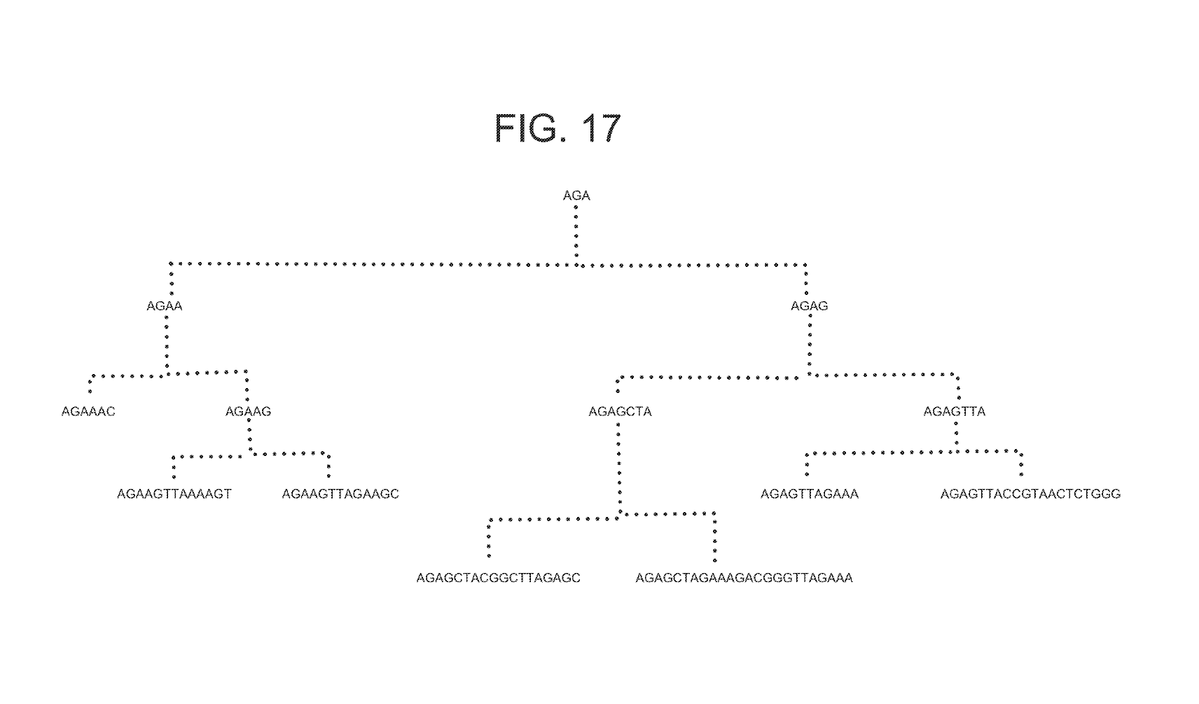

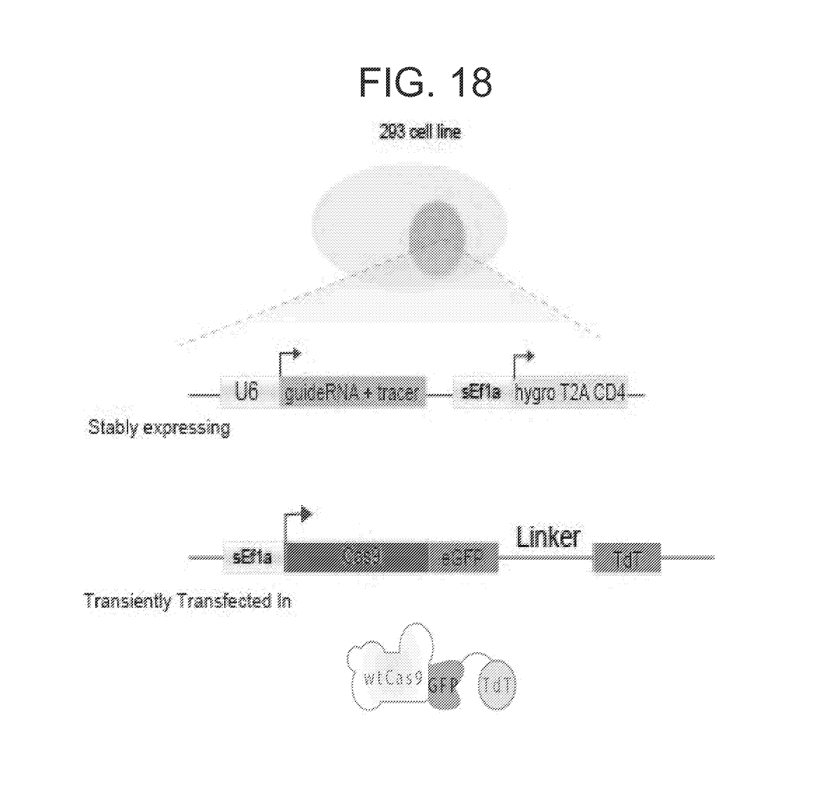

Terminal deoxynucleotidyl transferase (TdT) was determined to efficiently add nucleotides to a Cas9-induced dsDNA break. In these experiments, 293T cells were treated with either Cas9 or Cas9 and TdT as depicted in FIG. 18. In the absence of TdT, genomic deletions prevailed. In the presence of TdT, insertions were visualized by added nucleotides at the site of the dsDNA break. FIG. 16A displays dsDNA break at a conventional DNA locus. FIG. 16B displays a self-editing gRNA (segRNA) locus. Example sequencing results are displayed FIG. 17.

INFORMAL SEQUENCE LISTING

SEQ ID NO: 1
MDYKDDDDKDYKDDDDKMAPKKKRKVGIHGVPAADKKYSIGLDIGTNSVGWAVI
TDEYKVPSKKFKVLGNTDRHSIKKNLIGALLFDSGETAEATRLKRTARRRYTRRKNR
ICYLQEIFSNEMAKVDDSFFHRLEESFLVEEDKKHERHPIFGNIVDEVAYHEKYPTIYH
LRKKLVDSTDKADLRLIYLALAFIMIKFRGHFLIEGDLNPDNSDVDKLFIQLVQTYNQ
LFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGA
SQEEFYKFIKPILEKMDGTEELLVKLNREDLLRKQRTFDNGSIPHQIHLGELHAILRRQ
EDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKAQVS
GQGDSLHEHIANLAGSPAIKKGI
LQTVKVVDELVKVMGRHKPENIVIEMARENQTTQKGQKNSRERMKRIEEGIKELGS
QILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLICSKLV
SKFRKDFQFYKVREINNYHHAHDAYLNAVVGTALIKKYPKLESEFVYGDYKVYDVR
KMIAKSEQEIGKATAKYFFYSNIMNFFKTEITLANGEIRKRPUETNGETGEIVWDKGR
DFATVRKVL
SMPQVNrVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGG
DKRPAATKKAGQAKKKK
SEQ ID NO: 2 (WT guide RNA sequence):
GTTTTAGAGCTAGAAATAGCAAGTTAAAATAAGGCTAGTCCGTTATCAACTTGAA
AAAGTGGCACCGAGTCGGTGCTTTTTT
SEQ ID NO: 3 (GST-TAL-FokI-liker-FokI)
gcttaagcggtcgacggatcgggagatctcccgatcccctatggtgcactctcagtacaatctgctctgatgccgcatagttaagccagt
atctgctccctgcttgtgtgttggaggtcgctgagtagtgcgcgagcaaaatttaagctacaacaaggcaaggcttgaccgacaattgc
atgaagaatctgcttagggttaggcgttttgcgctgcttcgcgatgtacgggccagatatacgcgttgacattgattattgactagttattaa
tagtaatcaattacggggtcattagttcatagcccatatatggagttccgcgttacataacttacggtaaatggcccgcctggctgaccgc
ccaacgacccccgcccattgacgtcaataatgacgtatgttcccatagtaacgccaatagggactttccattgacgtcaatgggtggagt
atttacggtaaactgcccacttggcagtacatcaagtgtatcatatgccaagtacgccccctattgacgtcaatgacggtaaatggcccg
cctggcattatgcccagtacatgaccttatgggactttcctacttggcagtacatctacgtattagtcatcgctattaccatggtgatgcggt
tttggcagtacatcaatgggcgtggatagcggtttgactcacggggatttccaagtctccaccccattgacgtcaatgggagtttgttttg
gcaccaaaatcaacgggactttccaaaatgtcgtaacaactccgccccattgacgcaaatgggcggtaggcgtgtacggtgggaggt
ctatataagcagcgcgttttgcctgtactgggtctctctggttagaccagatctgagcctgggagctctctggctaactagggaacccact
gcttaagcctcaataaagcngccttgagtgcttcaagtagtgtgtgcccgtctgttgtgtgactctggtaactagagatccctca
tttagtcagtgtggaaaatctctagcagtggcgcccgaacagggacttgaaagcgaaagggaaaccagaggagctctctcgacgca
ggactcggcttgctgaagcgcgcacggcaagaggcgaggggcggcgactggtgagtacgccaaaaattttgactagcggaggcta
gaaggagagagatgggtgcgagagcgtcagtattaagcgggggagaattagatcgcgatgggaaaaaattcggttaaggccaggg
ggaaagaaaaaatataaattaaaacatatagtatgggcaagcagggagctagaacgattcgcagttaatcctggcctgttagaaacatc
agaaggctgtagacaaatactgggacagctacaaccatcccttcagacaggatcagaagaacttagatcattatataatacagtagcaa
ccctctattgtgtgcatcaaaggatagagataaaagacaccaaggaagctttagacaagatagaggaagagcaaaacaaaagtaaga
ccaccgcacagcaagcggccggccgcgctgatcttcagacctggaggaggagatatgagggacaattggagaagtgaattatataa
atataaagtagtaaaaattgaaccattaggagtagcacccaccaaggcaaagagaagagtggtgcagagagaaaaaagagcagtgg
gaataggagctttgttccttgggttcttgggagcagcaggaagcactatgggcgcagcgtcaatgacgctgacggtacaggccagac
aattattgtctggtatagtgcagcagcagaacaatttgctgagggctattgaggcgcaacagcatctgttgcaactcacagtctggggca
tcaagcagctccaggcaagaatcctggctgtggaaagatacctaaaggatcaacagctcctggggatttggggttgctctggaaaact
catttgcaccactgctgtgccttggaatgctagttggagtaataaatctctggaacagatttggaatcacacgacctggatggagtggga
cagagaaattaacaattacacaagcttaatacactccttaattgaagaatcgcaaaaccagcaagaaaagaatgaacaagaattattgg
aattagataaatgggcaagtttgtggaattggtttaacataacaaattggctgtggtatataaaattattcataatgatagtaggaggcttgg
taggtttaagaatagtttttgctgtactttctatagtgaatagagttaggcagggatattcaccattatcgtttcagacccacctcccaacccc
gaggggacccgacaggcccgaaggaatagaagaagaaggtggagagagagacagagacagatccattcgattagtgaacggatc
ggcactgcgtgcgccaattctgcagacaaatggcagtattcatccacaattttaaaagaaaaggggggattggggggtacagtgcag
gggaaagaatagtagacataatagcaacagacatacaaactaaagaattacaaaaacaaattacaaaaattcaaaattttcgggtttatta
cagggacagcagagatccagtttggttagtaccgggccctagagatcacgagactagcctcgagagatctgatcataatcagccatac
cacatttgtagaggttttacttgctttaaaaaacctcccacacctccccctgaacctgaaacataaaatgaatgcaattgttgttgttaacttg
tttattgcagcttataatggttacaaataaggcaatagcatcacaaatttcacaaataaggcatttttttcactgcattctagttttggtttgt
aaactcatcaatgtatcttatcatgtctggatctcaaatccctcggaagctgcgcctgtcatcgaattcctgcagcccggtgcatgactaa
gctagtaccggttaggatgcatgctagctcagttagcctcccccatctctcgacgcggccgctttacATGGTGAGCAAGG
GCGAGGAGCTGTTCACCGGGGTGGTGCCCATCCTGGTCGAGCTGGACGGCGACG
TAAACGGCCACAAGTTCAGCGTGTCCGGCGAGGGCGAGGGCGATGCCACCTACG
GCAAGCTGACCCTGAAGTTCATCTGCACCACCGGCAAGCTGCCCGTGCCCTGGCC
CACCCTCGTGACCACCCTGACCTACGGCGTGCAGTGCTTCAGCCGCTACCCCGAC
CACATGAAGCAGCACGACTTCTTCAAGTCCGCCATGCCCGAAGGCTACGTCCAG
GAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTC
AAGGAGGACGGCAACATCCTGGGGCACAAGCTGGAGTACAACTACAACAGCCA
CAACGTCTATATCATGGCCGACAAGCAGAAGAACGGCATCAAGGTGAACTTCAA
GATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAG
CACCCAGTCCGCCCTGAGCAAAGACCCCAACGAGAAGCGCGATCACATGGTCCT
GCTGGAGTTCGTGACCGCCGCCGGGATCACTCTCGGCATGGACGAGCTGTACAAg
gtggctcgagcggaggctggatcggtcccggtgtcttctatggaggtcaaaacagcgtggatggcgtctccaggcgatctgacggttc
actaaacgagctctgcttatataggcctcccaccgtacacgcctaccctcgagaagcttgatatcactagagctctagTGTGCCC
GTCAGTGGGCAGAGCGCACATCGCCCACAGTCCCCGAGAAGTTGGGGGGAGGGG
TCGGCAATTGAACCGGTGCCTAGAGAAGGTGGCGCGGGGTAAACTGGGAAAGTG
ATGTCGTGTACTGGCTCCGCCTTTTTCCCGAGGGTGGGGGAGAACCGTATATAAG
TGCAGTAGTCGCCGTGAACGTTCTTTTTCGCAACGGGTTTGCCGCCAGAACAgtgag
CTAGCgctaccggtcgccaccCCTAGGATGTCCCCTATACTAGGTTATTGGAAAATTAAGG
GCCTTGTGCAACCCACTCGACTTCTTTTGGAATATCTTGAAGAAAAATATGAAGA
GCATTTGTATGAGCGCGATGAAGGTGATAAATGGCGAAACAAAAAGTTTGAATT
GGGTTTGGAGTTTCCCAATCTTCCTTATTATATTGATGGTGATGTTAAATTAACAC
AGTCTATGGCCATCATACGTTATATAGCTGACAAGCACAACATGTTGGGTGGTTG
TCCAAAAGAGCGTGCAGAGAT1TCAATGCTTGAAGGAGCGGTTTTGGATATTAG
ATACGGTGTTTCGAGAATTGCATATAGTAAAGACTTTGAAACTCTCAAAGTTGAT
TTTCTTAGCAAGCTACCTGAAATGCTGAAAATGTTCGAAGATCGTTTATGTCATA
AAACATATTTAAATGGTGATCATGTAACCCATCCTGACTTCATGTTGTATGACGC
TCTTGATGTTGTTTTATACATGGACCCAATGTGCCTGGATGCGTTCCCAAAATTAG
TTTGTTTTAAAAAACGTATTGAAGCTATCCCACAAATTGATAAGTACTTGAAATC
CAGCAAGTATATAGCATGGCCTTTGCAGGGCTGGCAAGCCACGTTTGGTGGTGGC
GACCATCCTCCAAAATCGGATCTGGTTCCGCGTGGATCCGGCGGTAGTTTAAACat
ggcttcctcccctccaaagaaaaagagaaaggttagttggaaggacgcaagtggttggtctagagtggatctacgcacgctcggctac
agtcagcagcagcaagagaagatcaaaccgaaggtgcgttcgacagtggcgcagcaccacgaggcactggtgggccatgggttta
cacacgcgcacatcgttgcgctcagccaacacccggcagcgttagggaccgtcgctgtcacgtatcagcacataatcacggcgttgc
cagaggcgacacacgaagacatcgttggcgtcggcaaacagtggtccggcgcacgcgccctggaggcettgctcacggatgcgg
gggagttgagaggtccgccgttacagttggacacaggccaacttgtgaagattgcaaaacgtggcggcgtgaccgcaatggaggca
gtgcatgcatcgcgcaatgcactgacgggtgcccccctgaacCTGACCCCGGACCAAGTGGTGGCTATCG
CCAGCAACAATGGCGGCAAGCAAGCGCTCGAAACGGTGCAGCGGCTGTTGCCGG
TGCTGTGCCAGGACCATGGCCTGACCCCGGACCAAGTGGTGGCTATCGCCAGCA
ACGGTGGCGGCAAGCAAGCGCTCGAAACGGTGCAGCGGCTGTTGCCGGTGCTGT
GCCAGGACCATGGCCTGACCCCGGACCAAGTGGTGGCTATCGCCAGCAACAATG
GCGGCAAGCAAGCGCTCGAAACGGTGCAGCGGCTGTTGCCGGTGCTGTGCCAGG
ACCATGGCCTGACCCCGGACCAAGTGGTGGCTATCGCCAGCAACATTGGCGGCA
AGCAAGCGCTCGAAACGGTGCAGCGGCTGTTGCCGGTGCTGTGCCAGGACCATG
GCCTGACCCCGGACCAAGTGGTGGCTATCGCCAGCAACAATGGCGGCAAGCAAG
CGCTCGAAACGGTGCAGCGGCTGTTGCCGGTGCTGTGCCAGGACCATGGCCTGA
CTCCGGACCAAGTGGTGGCTATCGCCAGCCACGATGGCGGCAAGCAAGCGCTCG
AAACGGTGCAGCGGCTGTTGCCGGTGCTGTGCCAGGACCATGGCCTGACCCCGG
ACCAAGTGGTGGCTATCGCCAGCAACATTGGCGGCAAGCAAGCGCTCGAAACGG
TGCAGCGGCTGTTGCCGGTGCTGTGCCAGGACCATGGCCTGACTCCGGACCAAGT
GGTGGCTATCGCCAGCCACGATGGCGGCAAGCAAGCGCTCGAAACGGTGCAGCG
GCTGTTGCCGGTGCTGTGCCAGGACCATGGCCTGACTCCGGACCAAGTGGTGGCT
ATCGCCAGCCACGATGGCGGCAAGCAAGCGCTCGAAACGGTGCAGCGGCTGTTG
CCGGTGCTGTGCCAGGACCATGGCCTGACTCCGGACCAAGTGGTGGCTATCGCC
AGCCACGATGGCGGCAAGCAAGCGCTCGAAACGGTGCAGCGGCTGTTGCCGGTG
CTGTGCCAGGACCATGGCCTGACCCCGGACCAAGTGGTGGCTATCGCCAGCAAC
ATTGGCGGCAAGCAAGCGCTCGAAACGGTGCAGCGGCTGTTGCCGGTGCTGTGC
CAGGACCATGGCCTGACCCCGGACCAAGTGGTGGCTATCGCCAGCAACAATGGC
GGCAAGCAAGCGCTCGAAACGGTGCAGCGGCTGTTGCCGGTGCTGTGCCAGGAC
CATGGCCTGACTCCGGACCAAGTGGTGGCTATCGCCAGCCACGATGGCGGCAAG
CAAGCGCTCGAAACGGTGCAGCGGCTGTTGCCGGTGCTGTGCCAGGACCATGGC
CTGACCCCGGACCAAGTGGTGGCTATCGCCAGCAACAATGGCGGCAAGCAAGCG
CTCGAAACGGTGCAGCGGCTGTTGCCGGTGCTGTGCCAGGACCATGGCCTGACC
CCGGACCAAGTGGTGGCTATCGCCAGCAACAATGGCGGCAAGCAAGCGCTCGAA
ACGGTGCAGCGGCTGTTGCCGGTGCTGTGCCAGGACCATGGCCTGACCCCGGAC
CAAGTGGTGGCTATCGCCAGCAACATTGGCGGCAAGCAAGCGCTCGAAACGGTG
CAGCGGCTGTTGCCGGTGCTGTGCCAGGACCATGGCCTGACTCCGGACCAAGTG
GTGGCTATCGCCAGCCACGATGGCGGCAAGCAAGCGCTCGAAACGGTGCAGCGG
CTGTTGCCGGTGCTGTGCCAGGACCATGGCCTGACTCCGGACCAAGTGGTGGCTA
TCGCCAGCCACGATGGCGGCAAGCAAGCGCTCGAAACGGTGCAGCGGCTGTTGC
CGGTGCTGTGCCAGGACCATGGCCTGACCCCGGACCAAGTGGTGGCTATCGCCA
GCAACGGTGGCGGCAAGCAAGCGCTCGAAACGGTGCAGCGGCTGTTGCCGGTGC
TGTGCCAGGACCATGGCCTGACTCCGGACCAAGTGGTGGCTATCGCCAGCCACG
ATGGCGGCAAGCAAGCGCTCGAAACGGTGCAGCGGCTGTTGCCGGTGCTGTGCC
AGGACCATGGCCTGACCCCGGACCAAGTGGTGGCTATCGCCAGCCACGATGGCG
GCAAGCAAGCGCTCGAAACGGTGCAGCGGCTGTTGCCGGTGCTGTGCCAGGACC
ATGGCCTGACCCCGGACCAAGTGGTGGCTATCGCCAGCAACGGTGGCGGCAAGC
AAGCGCTCGAAACGGTGCAGCGGCTGTTGCCGGTGCTGTGCCAGGACCATGGCC
TGACTCCGGACCAAGTGGTGGCTATCGCCAGCCACGATGGCGGCAAGCAAGCGC
TCGAAACGGTGCAGCGGCTGTTGCCGGTGCTGTGCCAGGACCATGGCctgaccccggac
caagtggtggctatcgccagcaacggtggcggcaagcaagcgctcgaaagcattgtggcccagctgagccggcctgatccggcgtt
ggccgcgttgaccaacgaccacctcgtcgccttggcctgcctcggcggacgtcctgccatggatgcagtgaaaaagggattgccgc
acgcgccggaattgatcagaagagtcaatcgccgtattggcgaacgcacgtcccatcgcgttgcctctagatcccagCCTGCAG
GTTCCCAACTAGTCAAAAGTGAACTGGAGGAGAAGAAATCTGAACTTCGTCATA
AATTGAAATATGTGCCTCATGAATATATTGAATTAATTGAAATTGCCAGAAATTC
CACTCAGGATAGAATTCTTGAAATGAAGGTAATGGAATTTTTTATGAAAGTTTAT
GGATATAGAGGTAAACATTTGGGTGGATCAAGGAAACCGGACGGAGCAATTTAT
ACTGTCGGATCTCCTATTGATTACGGTGTGATCGTGGATACTAAAGCTTATAGCG
GAGGTTATAATCTGCCAATTGGCCAAGCAGATGAAATGCAACGATATGTCGAAG
AAAATCAAACACGAAACAAACATATCAACCCTAATGAATGGTGGAAAGTCTATC
CATCTTCTGTAACGGAATTTAAGTTTTTATTTGTGAGTGGTCACTTTAAAGGAAAC
TACAAAGCTCAGCTTACACGATTAAATCATATCACTAATTGTAATGGAGCTGTTC
TTAGTGTAGAAGAGCTTTTAATTGGTGGAGAAATGATTAAAGCCGGCACATTAAC
CTTAGAGGAAGTGAGACGGAAATTTAATAACGGCGAGATAAACTTTggcgcgcctggc
ggaggtggaagtgcaggtgctggatccggtagtggctcaggtggtggtggcggttcagctggcgctggaagtggttcaggtagtgg
aggaggaggcggctctgcaggagcaggctctggctccggatctggaggaggtggcggaagcgctggtgcaggctccggaagcg
gaagtggagcgatcgcttcccagctagtgaaatctgaattggaagagaagaaatctgaacttagacataaattgaaatatgtgccacat
gaatatattgaattgattgaaatcgcaagaaattcaactcaggatagaatccttgaaatgaaggtgatggagttctttatgaaggtttatggt
tatcgtggtaaacatttgggtggatcaaggaaaccagacggagcaatttatactgtcggatctcctattgattacggtgtgatcgttgatac
taaggcatattcaggaggttataatcttccaattggtcaagcagatgaaatgcaaagatatgtcgaagagaatcaaacaagaaacaagc
atatcaaccctaatgaatggtggaaagtctatccatcttcagtaacagaatttaagttcttgtttgtgagtggtcatttcaaaggaaactaca
aagctcagcttacaagattgaatcatatcactaattgtaatggagctgttcttagtgtagaagagcttttgattggtggagaaatgattaaag
ctggtacattgacacttgaggaagtgagaaggaaatttaataacggtgagataaactttTAGttaattaagaattcgtcgagggaccta
ataacttcgtatagcatacattatacgaagttatacatgtttaagggttccggttccactaggtacaattcgatatcaagcttatcgataatca
acctctggattacaaaatttgtgaaagattgactggtattcttaactatgttgctccttttacgctatgtggatacgctgctttaatgcctttgtat
catgctattgcttcccgtatggctttcattttctcctccttgtataaatcctggttgctgtctctttatgaggagttgtggcccgttgtcaggcaa
cgtggcgtggtgtgcactgtgtttgctgacgcaacccccactggttggggcattgccaccacctgtcagctcctttccgggactttcgctt
tccccctccctattgccacggcggaactcatcgccgcctgccttgcccgctgctggacaggggctcggctgttgggcactgacaattc
cgtggtgttgtcggggaaatcatcgtcctttccttggctgctcgcctgtgttgccacctggattctgcgcgggacgtccttctgctacgtcc
cttcggccctcaatccagcggaccttccttcccgcggcctgctgccggctctgcggcctcttccgcgtcttcgccttcgccctcagacg
agtcggatctccctttgggccgcctccccgcatcgataccgtcgacctcgatcgagacctagaaaaacatggagcaatcacaagtagc
aatacagcagctaccaatgctgattgtgcctggctagaagcacaagaggaggaggaggtgggttttccagtcacacctcaggtaccttt
aagaccaatgacttacaaggcagctgtagatcttagccactttttaaaagaaaaggggggactggaagggctaattcactcccaacga
agacaagatatccttgatctgtggatctaccacacacaaggctacttccctgattggcagaactacacaccagggccagggatcagata
tccactgacctttggatggtgctacaagctagtaccagttgagcaagagaaggtagaagaagccaatgaaggagagaacacccgctt
gttacaccctgtgagcctgcatgggatggatgacccggagagagaagtattagagtggaggtttgacagccgcctagcatttcatcac
atggcccgagagctgcatccggactgtactgggtctctctggttagaccagatctgagcctgggagctctctggctaactagggaacc
cactgcttaagcttcaataaagcttgccttgagtgcttcaagtagtgtgtgcccgtctgttgtgtgactctggtaactagagatccctcagt
cccttttagtcagtgtggaaaatctctagcagcatgtgagcaaaaggccagcaaaaggccaggaaccgtaaaaaggccgcgttgctg
gcgtttttccataggctccgcccccctgacgagcatcacaaaaatcgacgctcaagtcagaggtggcgaaacccgacaggactataa
agataccaggcgtttccccctggaagctccctcgtgcgctctcctgttccgaccctgccgcttaccggatacctgtccgcctttctccctt
cgggaagcgtggcgctttctcatagctcacgctgtaggtatctcagttcggtgtaggtcgttcgctccaagctgggctgtgtgcacgaac
cccccgttcagcccgaccgctgcgccttatccggtaactatcgtcttgagtccaacccggtaagacacgacttatcgccactggcagca
gccactggtaacaggattagcagagcgaggtatgtaggcggtgctacagagttcttgaagtggtggcctaactacggctacactagaa
gaacagtatttggtatctgcgctctgctgaagccagttaccttcggaaaaagagttggtagctcttgatccggcaaacaaaccaccgctg
gtagcggtggtttttttgtttgcaagcagcagattacgcgcagaaaaaaaggatctcaagaagatcctttgatcttttctacggggtct
gctcagtggaacgaaaactcacgttaagggattttggtcatgagattatcaaaaaggatcttcacctagatccttttaaattaaaaatgaag
ttttaaatcaatctaaagtatatatgagtaaacttggtctgacagttaccaatcttaatcagtgaggcacctatctcagcgatctgtctatttc
gttcatccatagttgcctgactccccgtcgtgtagataactacgatacgggagggcttaccatctggccccagtgctgcaatgataccgc
gagacccacgctcaccggctccagatttatcagcaataaaccagccagccggaagggccgagcgcagaagtggtcctgcaactttat
ccgcctccatccagtctattaattgttgccgggaagctagagtaagtagttcgccagttaatagtttgcgcaacgttgttgccattgctaca
ggcatcgtggtgtcacgctcgtcgtttggtatggcttcattcagctccggttcccaacgatcaaggcgagttacatgatcccccatgttgt
gcaaaaaagcggttagctccttcggtcctccgatcgttgtcagaagtaagttggccgcagtgttatcactcatggttatggcagcactgc
ataattctcrtactgtcatgccatccgtaagatgcttttctgtgactggtgagtactcaaccaagtcattctgagaatagt
cgagttgctcttgcccggcgtcaatacgggataataccgcgccacatagcagaactttaaaagtgctcatcattggaaaacgttcttcgg
ggcgaaaactctcaaggatcttaccgctgttgagatccagttcgatgtaacccactcgtgcacccaactgatcttcagcatcttttactttc
accagcgtttctgggtgagcaaaaacaggaaggcaaaatgccgcaaaaaagggaataagggcgacacggaaatgttgaatactcat
actcttcctttttcaatattattgaagcatttatcagggttattgtctcatgagcggatacatatttgaatgtatttagaaaaataaacaaatagg
ggttccgcgcacatttccccgaaaagtgccacctgac
SEQ ID NO: 4: (Linker)
CCTAGGGGGGGAGGGTCCGGCGGCGGTTCCGGCGGAGGATCGGGTGGAGGGTCA
GGTGGAGGCTCAGGCGGTGGATCAGGAGGAGGGAGCGGTGGCGGGAGCGGCGG
AGGGTCGGGAGGAGGTTCGGGCGGAGGCTCGGGCGGTGGGTCCGGAGGTGGCTC
GGGAGGCGGAAGCGGAGGCGGGTCCGGTGGCGGATCAGGCGGAGGCAGCGGAG
GAGGATCAGGTGGCGGAAGCGGAGGCGGCTCCGGAGGAGGCTCCGGCGGTGGA
AGCGGTGGAGGAAGCGGCGGCGGATCGGGAGGTGGGTCG
SEQ ID NO: 5: (Protein sequence of linker)
PRGGGSGGGSGGGSGGGSGGGSGGGSGGGSGGGSGGGSGG
GSGGGSGGGSGGGSGGGSGGGSGGGSGGGSGGGSGGGSGG
GSGGGSGGGSGGGSGGGSGGGS
SEQ ID NO: 6: (Linker sequence)
ggcggaggtggaagtgcaggtgctggatccggtagtggctcaggtggtggtggcggttcagctggcgctggaagtggttcaggtag
tggaggaggaggcggctctgcaggagcaggctctggctccggatctggaggaggtggcggaagcgctggtgcaggctccggaag
cggaagtgga
SEQ ID NO: 7: (linker protein sequence)
GGGGSAGAGSGSGSGGGGGSAGAGSGSGSGGGGGSAGAGS
GSGSGGGGGSAGAGSGSGSG

### REFERENCES

1 Sakaue-Sawano, A. et al. Visualizing spatiotemporal dynamics of multicellular cell-cycle progression. Cell 132, 487-498, doi:10.1016/j.cell.2007.12.033 (2008).
2 Ke, R. et al. In situ sequencing for RNA analysis in preserved tissue and cells. Nat Methods 10, 857-860, doi:10.1038/nmeth.2563 (2013).
3 Mino, T., Aoyama. Y. & Sera, T. Efficient double-stranded DNA cleavage by artificial zinc-finger nucleases composed of one zinc-finger protein and a single-chain FokI dimer. Journal of biotechnology 140, 156-161, doi:10.1016/j.jbiotec.2009.02.004 (2009).
4 Komori, T., Okada, A., Stewart, V. & Alt, F. W. Lack of N regions in antigen receptor variable region genes of TdT-deficient lymphocytes. Science 261, 1171-1175 (1993).
5 Boubakour-Azzouz, I., Bertrand, P., Claes, A., Lopez, B. S. & Rougeon, F. Terminal deoxynucleotidyl transferase requires KU80 and XRCC4 to promote N-addition non-V(D)J chromosomal breaks in non-lymphoid cells. Nucleic Acids Res 40, 8381-8391, doi:10.1093/nar/gks585 (2012).

6 Eastburn, D. J., Sciambi, A. & Abate, A. R. Ultrahigh-throughput Mammalian single-cell reverse-transcriptase polymerase chain reaction in microfluidic drops. Anal Chem 85, 8016-8021, doi:10.1021/ac402057q (2013).
Vogt W . . . . Vitalfiirbung. II. Teil. Gastrulation und Mesodermbildung bei Urodelen und Anuren. W. Roux Arch Entwicklungsmech Org 120384-706. Keller R E (1986) . . . Developmental Biology; 1929.
Sulston J E, Schierenberg E, White J G, Thomson J N. The embryonic cell lineage of the nematode Caenorhabditis elegans. Developmental Biology. 1983 November; 100(1):64-119.
Livet J, Weissman T A, Kang H, Draft R W, Lu J. Transgenic strategies for combinatorial expression of fluorescent proteins in the nervous system. Nature. 2007.
Snippert H J, van der Flier Sato T, van Es J H, van den Born M, Kroon-Veenboer C, et al. Intestinal Crypt Homeostasis Results from Neutral Competition between Symmetrically Dividing Lgr5 Stem Cells. Cell. 2010 October; 143(1):134-44.
Mino T, Aoyama Y, Sera T. Efficient double-stranded DNA cleavage by artificial zinc-finger nucleases composed of one zinc-finger protein and a single-chain FokI dimer, Journal of Biotechnology. 2009 March; 140(3-4):156-61.
Sakaue-Sawano A, Kurokawa H, Morimura ‘1’, Hanyu A, Hama. H, Osawa H, et al. Visualizing Spatiotemporal Dynamics of Multicellular Cell-Cycle Progression. Cell. 2008 February; 132(3):487-98.
Ke R, Mignardi M, Pacureanu A, Svedlund, J, Botling J, C, et al. In situ sequencing for RNA analysis in preserved tissue and cells. Nature methods, 2013 September; 10(9):857-60.
Batzer M A, Gudi V A, Mena J C, Foltz D W, Herrera R J, Deininger P L. Amplification dynamics of human-specific (HS) alu family members. Nucleic Acids Res. Oxford University Press; 1991 July 11; 19(13):3619-23.
Ohtsuka E, Matsuki S, Ikehara M, Takahashi Y, Matsubara. K. An alternative approach to deoxyoligonucleotides as hybridization probes by insertion of deoxyinosine at ambiguous codon positions. Journal of Biological Chemistry. American Society for Biochemistry and Molecular Biology; 1985 March 10; 260(5):2605-8.
Rossolini G M, Cresti S, Ingianni A, Cattani P, Riccio M L, Satta G. Use of deoxyinosine-containing primers vs degenerate primers for polymerase chain reaction based on ambiguous sequence information. Molecular and Cellular Probes. 1994 April; 8(2):91-8.
Maratea D, Young K, Young R. Deletion and fusion analysis of the phage φX174 lysis gene. E. Gene. 1985 January; 40(1):39-46.
Murphy J R, Bishai W, Borowski M, Miyanohara A, Boyd J, Nagle S. Genetic construction, expression, and melanoma-selective cytotoxicity of a diphtheria toxin-related alpha-melanocyte-stimulating hormone fission protein. Proc Natl Acad Sci USA. National Acad Sciences; 1986 November; 83(20):8258-62.
Kwoh D Y, Davis G R, Whitfield K M, Chappelle H L, DiMichele L J, Gingeras T R. Transcription-based amplification system and detection of amplified human immunodeficiency virus type 1 with a bead-based sandwich hybridization format. Proc Natl. Acad Sci USA. National Acad Sciences; 1989 February; 86(4):1173-7.
Guatelli J C, Whitfield K M, Kwoh D Y, Barringer K J, Richman D D, Gingeras T R. Isothermal, in vitro amplification of nucleic acids by a multienzyme reaction modeled after retroviral replication. Proc Natl Acad Sci USA. National Acad Sciences; 1990 March; 87(5): 1874-8.
Lomeli H, Tyagi S, Pritchard C G, Lizardi P M, Kramer F R. Quantitative assays based on the use of replicatable hybridization probes. Clinical Chemistry. American Association for Clinical Chemistry; 1989 September; 35(9):1826-11,
Landegren U, Kaiser R, Sanders J, Hood L. A ligase-mediated gene detection technique. Science. American Association for the Advancement of Science; 1988 August 26; 241(4869):1077-80.
Wu D Y, Wallace R B. The ligation amplification reaction (LAR)—Amplification of specific DNA sequences using sequential rounds of template-dependent ligation. Genomics. 1989 May; 4(4):560-9.
Barringer K J, Orgel L, Wahl G, Gingeras T R. Blunt-end and single-strand ligations by Escherichia coli ligase: influence on an in vitro amplification scheme. Gene. 1990 April; 89(1):117-22,
Jiménez J I, Xulvi-Brunet R, Campbell G W, Turk-MacLeod R, Chen I A. Comprehensive experimental fitness landscape and evolutionary network for small RNA. Proc Natl Acad Sci USA. National Acad Sciences; 2013 September 10; 110(37):14984-9.
Schloss P D, Westcott S L, Ryabin T, Hall I R, Hartmann M, Hollister E B, et al. Introducing mothur: open-source, platform-independent, community-supported software for describing and comparing microbial communities. Appl Environ Microbiol. American Society for Microbiology; 2009 December; 75(23):7537-41.

Li W, Godzik A. Cd-hit: a fast program for clustering and comparing large sets of protein or nucleotide sequences. Bioinformatics. 2006.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A method of forming a barcoded cell said method comprising,
(i) expressing in a cell a heterologous cleaving protein complex comprising a sequence-specific DNA-binding domain and a nucleic acid cleaving domain; wherein said sequence-specific DNA-binding domain targets said nucleic acid cleaving domain to a genomic nucleic acid sequence, thereby forming a genomic nucleic acid sequence bound to said heterologous cleaving protein complex;
(ii) introducing a double-stranded cleavage site in said genomic nucleic acid sequence bound to said heterologous cleaving protein complex, thereby forming a double-stranded cleavage site in said genomic nucleic acid sequence; and
(iii) inserting random nucleotides at said double-stranded cleavage site, thereby forming said barcoded cell.

2. The method of claim 1, further comprising after said inserting step in (iii):
(iv) allowing said barcoded cell to divide, thereby forming a barcoded progeny of cells;
(v) collecting said barcoded progeny;
(vi) nucleotide sequencing said barcoded nucleic acid sequence; and
(vii) correlating said barcoded nucleic acid sequence.

3. The method of claim 1 or 2, further comprising after said inserting step in (iii) and before said allowing step in (iv), (iii.i) ligating the ends of said double-stranded cleavage site.

4. The method of any one of the preceding claims, wherein said sequence-specific DNA-binding domain comprises an RNA molecule.

5. The method of claim 4, wherein said RNA molecule is a guide RNA.

6. The method of claim 4, wherein said RNA molecule comprises a nucleic acid cleaving domain recognition site.

7. The method of any one of claims 1 to 6, wherein said nucleic acid cleaving domain comprises a Cas9 domain or functional portion thereof.

8. The method any one of claims 1 to 7, wherein said genomic nucleic acid sequence comprises a guide RNA encoding sequence.

9. The method of claim 1 or 2, wherein said sequence-specific DNA-binding domain is a TAL effector DNA binding domain or functional portion thereof.

10. The method of claim 1 or 2, wherein said sequence-specific DNA-binding domain is a zinc finger domain or functional portion thereof.

11. The method of claim 9 or 10, wherein said nucleic acid cleaving domain comprises a restriction enzyme or functional portion thereof.

12. The method of claim 11, wherein said restriction enzyme is MmeI or FokI.

13. The method of any one of the preceding claims, wherein said inserting comprises targeting a recombinant DNA editing protein to said double-stranded cleavage site.

14. The method of any one of claims 1-12, wherein said inserting comprises targeting an endogenous DNA editing protein to said double-stranded cleavage site.

15. The method of claim 13, wherein said recombinant DNA editing protein is a heterologous DNA editing protein.

16. The method of claim 15, wherein said recombinant DNA editing protein comprises a sequence-specific DNA-binding domain and a terminal deoxynucleotidyl transferase (TdT) domain.

17. The method of claim 16, wherein said sequence-specific DNA-binding domain is a TAL effector DNA binding domain or functional portion thereof.

18. The method of claim 16, wherein said sequence-specific DNA-binding domain is a zinc finger domain or functional portion thereof.

19. A recombinant cleaving ribonucleoprotein complex comprising,
(i) a sequence-specific DNA-binding RNA molecule; and
(ii) a nucleic acid cleaving domain; wherein said RNA molecule comprises a nucleic acid cleaving domain recognition site.

20. The recombinant cleaving ribonucleoprotein complex of claim 19, wherein said RNA molecule is a guide RNA.

21. The recombinant cleaving ribonucleoprotein complex of claim 19, wherein said RNA molecule comprises a nucleic acid cleaving domain recognition site.

22. The recombinant cleaving ribonucleoprotein complex of any one of claims 19 to 21, wherein said nucleic acid cleaving domain comprises a Cas9 domain or functional portion thereof.

23. The recombinant cleaving ribonucleoprotein complex of any one of claims 19 to 22, further comprising a recombinant DNA editing protein.

24. The recombinant cleaving ribonucleoprotein complex of claim 23, wherein said recombinant DNA editing protein comprises a terminal deoxynucleotidyl transferase domain.

25. The recombinant cleaving ribonucleoprotein complex of claim 23, wherein said recombinant DNA editing protein comprises a sequence-specific DNA-binding domain.

26. A nucleic acid encoding a recombinant cleaving ribonucleoprotein complex of any one of claims 19-25.

27. A cell comprising the nucleic acid of claim 26.

28. The cell of claim 27, further comprising a promoter operably linked to the nucleic acid.

29. A non-human animal comprising the cell of claim 27 or 28.

30. A method of forming a barcoded cell said method comprising:
(i) expressing in a cell a recombinant cleaving ribonucleoprotein complex of any one of claims 19-25; wherein said sequence-specific DNA-binding RNA molecule targets said nucleic acid cleaving domain to a genomic nucleic acid sequence, thereby forming a genomic nucleic acid sequence bound to said recombinant cleaving ribonucleoprotein complex;
(ii) introducing a double-stranded cleavage site in said genomic nucleic acid sequence bound to said recombinant cleaving ribonucleoprotein complex, thereby forming a double-stranded cleavage site in said genomic nucleic acid sequence; and
(iii) targeting said recombinant DNA editing protein to said double-stranded cleavage site such as said recombinant DNA editing protein inserts a barcoded nucleic acid sequence into said double-stranded cleavage site; thereby forming said barcoded cell.

31. The method of claim 30, further comprising after said targeting step in (iii):
(iv) allowing said barcoded cell to divide, thereby forming a barcoded progeny of cells;
(v) collecting said barcoded progeny;
(vi) nucleotide sequencing said barcoded nucleic acid sequence; and
(vii) correlating said barcoded nucleic acid sequence.

32. The method of claim 30 or 31, further comprising after said inserting step in (iii) and before said allowing step in (iv), (iii.i) ligating the ends of said double-stranded cleavage site.

33. A recombinant DNA editing protein comprising:
(i) a sequence-specific DNA-binding domain; and
(ii) a terminal deoxynucleotidyl transferase domain.

34. The recombinant DNA editing protein of claim 33, wherein said sequence-specific DNA-binding domain comprises an RNA molecule.

35. The recombinant DNA editing protein of claim 34, wherein said RNA molecule is a guide RNA.

36. The recombinant DNA editing protein of claim 34, wherein said RNA molecule comprises a nucleic acid cleaving domain recognition site.

37. The recombinant DNA editing protein of claim 33, wherein said sequence-specific DNA-binding domain is a TAL effector DNA binding domain or functional portion thereof.

38. The recombinant DNA editing protein of claim 37, wherein said sequence-specific DNA-binding domain is a zinc finger domain or functional portion thereof.

39. The recombinant DNA editing protein of any one of claims 33 to 38, further comprising a nucleic acid cleaving domain.

40. The recombinant DNA editing protein of claim 39, wherein said nucleic acid cleaving domain is a restriction enzyme.

41. The recombinant DNA editing protein of claim 40, wherein said restriction enzyme is MmeI or FokI.

42. A nucleic acid encoding a recombinant cleaving protein of any one of claims 43-41.

43. A recombinant cleaving protein comprising:
(i) a cell cycle regulated domain;
(ii) a sequence-specific DNA-binding domain; and
(iii) a DNA cleaving domain;
wherein said cell cycle regulated domain is operably linked to one end of said sequence-specific DNA-binding domain and said DNA cleaving domain is linked to the other end of said sequence-specific DNA-binding domain.

44. The recombinant cleaving protein of claim 1, wherein all of said domains are heterologous to each other.

45. The recombinant cleaving protein of claim 1, wherein said cell cycle regulated domain is a peptide domain.

46. The recombinant cleaving protein of claim 45, wherein said peptide domain is a Geminin peptide.

47. The recombinant cleaving protein of claim 1, wherein said sequence-specific DNA-binding domain is TAL effector DNA binding domain.

48. The recombinant cleaving protein of claim 1, wherein said DNA cleaving domain comprises a cleaving agent dimer.

49. The recombinant cleaving protein of claim 48, wherein said cleaving agent dimer comprises a first cleaving agent and a second cleaving agent.

50. The recombinant cleaving protein of claim 49, wherein said first cleaving agent and said second cleaving agent are linked through a linker.

51. The recombinant cleaving protein of claim 50, wherein said first cleaving agent and said second cleaving agent are a FokI nuclease.

52. The recombinant cleaving protein of claim 50, wherein said first cleaving agent and said second cleaving agent are a MmeI nuclease.

53. A nucleic acid encoding a recombinant cleaving protein of any one of claims 43-52.

54. A recombinant DNA editing protein comprising:
(i) a cell cycle regulated domain;
(ii) a sequence-specific DNA-binding domain; and
(iii) a terminal deoxynucleotidyl transferase domain;
wherein said cell cycle regulated domain is operably linked to one end of said sequence-specific DNA-binding domain and said terminal deoxynucleotidyl transferase domain is linked to the other end of said sequence-specific DNA-binding domain.

55. A nucleic acid encoding a recombinant DNA editing protein of claim 54.

56. A cell comprising a recombinant cleaving protein of any one of claims 43-52, a recombinant DNA editing protein of claim 54 or both.

57. The cell of claim 56, wherein said cell is a zygote.

58. The cell of claim 56, wherein said cell forms part of an organism.

59. A method of forming a barcoded cell said method comprising:
(i) expressing in a cell a recombinant cleaving protein and a recombinant DNA editing protein in a cell cycle-dependent manner;
(ii) targeting said recombinant cleaving protein to a genomic nucleic acid sequence, thereby introducing a double-stranded cleavage site in said genomic nucleic acid sequence;
(iii) targeting said recombinant DNA editing protein to said double-stranded cleavage site such as said recombinant DNA editing protein inserts a barcoded nucleic acid sequence into said double-stranded cleavage site; thereby forming said barcoded cell.

60. A method of forming a barcoded cell said method comprising:
(i) expressing in a cell a recombinant cleaving protein of any one of claims 43-52 and a recombinant DNA editing protein of claim 54 in a cell cycle-dependent manner;
(ii) targeting said recombinant cleaving protein to a genomic nucleic acid sequence, thereby introducing a double-stranded cleavage site in said genomic nucleic acid sequence;
(iii) targeting said recombinant DNA editing protein to said double-stranded cleavage site such as said recombinant DNA editing protein inserts a barcoded nucleic acid sequence into said double-stranded cleavage site; thereby forming said barcoded cell.

61. The method of claim 59 or 60, further comprising after said targeting step in (iii):
(iv) allowing said barcoded cell to divide, thereby forming a barcoded progeny of cells;
(v) collecting said barcoded progeny;
(vi) nucleotide sequencing said barcoded nucleic acid sequence; and
(vii) correlating said barcoded nucleic acid sequence.

62. The method of claim 59 or 60, wherein said expressing in a cell cycle dependent manner comprises expressing in S, G1, or M phase.

63. The method of claim 59 or 60, further comprising after said inserting step in (iii), ligating the ends of said double-stranded cleavage site.
